(19) United States
(12) Patent Application Publication
(10) Pub. No.: US 2023/0034337 A1
(43) Pub. Date: Feb. 2, 2023

(54) ANIMAL DATA PREDICTION SYSTEM

(71) Applicant: SPORTS DATA LABS, INC., Royal Oak, MI (US)

(72) Inventors: Mark GORSKI, Royal Oak, MI (US); Vivek KHARE, Cupertino, CA (US); Stanley MIMOTO, Bethel Island, CA (US)

(73) Assignee: SPORTS DATA LABS, INC., Royal Oak, MI (US)

(21) Appl. No.: 16/977,278

(22) PCT Filed: Apr. 15, 2020
(86) PCT No.: PCT/US2020/028313

§ 371 Date: Sep. 1, 2020

Related U.S. Application Data
(60) Provisional application No. 62/833,970, filed on Apr. 15, 2019.
(60) Provisional application No. 62/912,822, filed on Oct. 9, 2019.

(51) Int. Cl. G06Q 30/02; G06F 16/26; G06Q 50/34; G07F 17/32

(52) U.S. Cl. CPC G06Q 30/0202; G06F 16/26; G06Q 50/34; G07F 17/3288; G07F 17/323

(57) ABSTRACT

A speculation system for providing animal data and predictive indicators thereof includes one or more source sensors that collect animal data from targeted individuals. Characteristically, the animal data can be transmitted electronically. A computing subsystem receives the animal data. At least a portion of the animal data is transformed by the computing subsystem or the one or more sensors into a predictive indicator for a selected targeted individual or a group of targeted individuals. The computing subsystem operable to provide the predictive indicator and optionally at least a portion of the animal data to a user. Advantageously, a transmission subsystem providing transmission of the animal data to the computing subsystem.

### CROSS-REFERENCE TO RELATED APPLICATIONS

This application claims the benefit of U.S. provisional application Ser. No. 62/833,970 filed Apr. 15, 2019 and U.S. provisional application Ser. No. 62/912,822 filed Oct. 9, 2019, the disclosures of which are hereby incorporated in their entirety by reference herein.

### TECHNICAL FIELD

In at least one aspect, the present invention is related to systems for information from animal data to make predictions.

### BACKGROUND

The continuing advances in the availability of information over the Internet has substantially changed the way that business is conducted. Simultaneous with this information explosion, sensor technology, and in particular, biosensor technology has also progressed. In particular, miniature biosensors that measure electrocardiogram signals, blood flow, body temperature, perspiration levels, or breathing rate are now available. The ability of data from such sensors to be transmitted wirelessly and over the Internet has opened up potential new applications for data set collections.

Accordingly, there is a need for systems that collect, organize, and analyze sensor data for new applications, including, but not limited to, wagering and probability assessment systems.

### SUMMARY

In at least one aspect, a speculation system for providing animal data and predictive indicators thereof is provided. The speculation system includes one or more source sensors that collect animal data from one or mom targeted individuals. Characteristically, the animal data can be transmitted wirelessly or with a wired connection. A computing subsystem receives animal data with at least a portion of the animal data being transformed by the computing subsystem or the one or more source sensors into at least one computed asset assigned to a selected targeted individual or a group of targeted individuals. The one or more source sensors or the computing subsystem are operable to transform the at least one computed asset into a predictive indicator, and provide the predictive indicator, the at least one computed asset, and/or at least a portion of the animal data to one or more users. A transmission subsystem transmits at least a portion of the animal data to the computing subsystem.

### BRIEF DESCRIPTION OF THE DRAWINGS

FIG. 1 provides a schematic illustration of a speculation system that provides animal data along with a predictive indicator to a user.

FIG. 2 provides a schematic of example transmission subsystems that can be used in the system of FIG. 1.

FIG. 3 provides a schematic of a speculation system applied to wagering in sports.

FIG. 4 provides an example of a source page for an advertisement that can be displayed in an Iframe.

FIGS. 5A, 5B, 5C, SD, 5E, 5F, and 5G provide examples of advertisements in an Iframe for opportunities related to the speculation system.

FIG. 6 provides an example of a medium for user consumption, e.g., pop-up or embedded media that may be displayed when a user is viewing media such as a live sporting event while soliciting a user to place one or more bets.

FIG. 7 is an example of a homepage of a wagering application.

FIG. 8 provides an example of a wagering interface that a user would visit to evaluate one or more probabilities and/or place one or more bets.

FIG. 9 provides examples of the types of markets that can be created using outputs from the speculation system (labeled “Human Data Bets” for clarity purposes).

FIG. 10 provides an example when a user chooses a new market for placing a bet.

FIG. 11 provides an example of a health monitoring interface that can be created using one or more outputs from the speculation system.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and % or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

While the terms “probability” and “odds” are mathematically different (e.g., “probability” can be defined as the number of occurrences of a certain event expressed as a proportion of all events that could occur, whereas “odds” can be defined as the number of occurrences of a certain event expressed as a proportion of the number of non-occurrences of that event), both describe the likeliness that an event will occur. They are used interchangeably to avoid redundancy, and reference to one term should be interpreted to mean reference to both.

With respect to the terms “bet” and “wager,” both terms mean an act of taking a risk (e.g., money, non-financial consideration) on the outcome of a future event. Risk includes both financial (e.g., monetary) and non-financial risk (e.g., health, life). A risk can be taken against another one or more parties (e.g., an insurance company deciding whether to provide insurance) or against oneself (e.g., an individual deciding whether to obtain insurance), on the basis of an outcome, or the likelihood of an outcome, of a future event. The act of making a “bet” or “wager” can occur within or as part of any system or subsystem where one or more risks can be taken, including any system where a risk is gamified (e.g., gambling, sports betting). Where the terms “bet” or “wager” are used herein, the presently disclosed and claimed subject matter can use either of the other two terms interchangeably.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

When a computing device is described as performing an action or method step, it is understood that the computing device is operable to perform the action or method step typically by executing one or more lines of source code. The actions or method steps can be encoded onto non-transitory memory (e.g., hard drives, optical drive, flash drives, and the like).

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” and “multiple” as a subset.

Throughout this application, where publications are referenced, the disclosures of these publications in their entireties are hereby incorporated by reference into this application to more fully describe the state of the art to which this invention pertains.

The term “server” refers to any computer or computing device (including, but not limited to, desktop computer, notebook computer, laptop computer, mainframe, mobile phone, smart watches/glasses, AR/VR headset, and the like), distributed system, blade, gateway, switch, processing device, or combination thereof adapted to perform the methods and functions set forth herein.

The term “computing device” refers generally to any device that can perform at least one function, including communicating with another computing device. In a refinement, a computing device includes a central processing unit that can execute program steps and memory for storing data and a program code. As used herein, a computing subsystem is a computing device.

The processes, methods, or algorithms disclosed herein can be deliverable to/implemented by a computing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, other magnetic and optical media, and shared or dedicated cloud computing resources. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

The term “subject” or “individual” are synonymous and refer to a human or other animal, including birds and fish as well as all mammals including primates (particularly higher primates), horses, sheep, dogs, rodents, guinea pigs, cats, whales, rabbits, and cows. The one or more subjects may be, for example, humans participating in athletic training or competition, horses racing on a track, humans playing a video game, humans monitoring their personal health, humans providing their data to a third party, humans participating in a research or clinical study, or humans participating in a fitness class. A subject or individual can also be a derivative of a human or other animal (e.g., lab-generated organism derived at least in part from a human or other animal), one or more individual components, elements, or processes of a human or other animal that comprise the human or other animal (e.g., cells, proteins, biological fluids, amino acid sequences, tissues, hairs, limbs), or one or more artificial creations that share one or more characteristics with a human or other animal (e.g., lab-grown human brain cells that produce an electrical signal similar to that of human brain cells). In a refinement, the subject or individual can be a machine (e.g., robot, autonomous vehicle, mechanical arm) or network of machines programmable by one or more computing devices that share at least one biological function with a human or other animal and from which one or more types of biological data can be derived, which may be, at least in part, artificial in nature (e.g., data from artificial intelligence-derived activity that mimics biological brain activity).

The term “animal data” refers to any data obtainable from, or generated directly or indirectly, by a subject that can be transformed into a form that can be transmitted to a server or other computing device. Typically, the animal data is electronically transmitted with a wired or wireless connection. Animal data includes any data that can be obtained from one or more sensors or sensing equipment/systems, and in particular, biological sensors (biosensors). Animal data can also include descriptive data, auditory data, visually-captured data, neurologically-generated data (e.g., brain signals from neurons), data that can be manually entered related to a subject (e.g., medical history, social habits, feelings of a subject), and data that includes at least a portion of real animal data. In a refinement, the term “animal data” is inclusive of any derivative of animal data. In another refinement, animal data includes metadata gathered with the animal data. In another refinement, animal data includes at least a portion of simulated data. In yet another refinement, animal data is inclusive of simulated data.

The term “insight” refers to one or more descriptions that can be assigned to a targeted individual that describe a condition or status of the targeted individual. Examples include descriptions of stress levels (e.g., high stress, low stress), energy levels, fatigue levels, and the like. Insights may be quantified by one or more numbers or a plurality of numbers and may be represented as a probability or similar odds-based indicator. Insights may also be characterized by one or more other metrics or indices of performance that are predetermined (e.g., visually such as a color or physically such as a vibration).

The term “computed asset” refers to one or more numbers, a plurality of numbers, values, metrics, readings, insights, graphs, charts, or plots that are derived from at least a portion of the animal data or its one or more derivatives. The one or more sensors used herein initially provide an electronic signal. The computed asset is extracted or derived, at least in part, from the one or more electronic signals or its one or more derivatives. The computed asset describes or quantifies an interpretable property of the one or more targeted individuals. For example, electrocardiogram readings can be derived from analog front end signals (the electronic signal from the sensor), heart rate data (e.g., heart rate beats per minute) can be derived from electrocardiogram or PPG sensors, body temperature data can be derived from temperature sensors, perspiration data can be derived from perspiration sensors, glucose information can be derived from biological fluid sensors, DNA and RNA sequencing information can be derived from sensors that obtain genomic and genetic data, brain activity data can be derived from neurological sensors, hydration data can be derived from in-mouth saliva sensors, location data can be derived from GPS or RFID sensors, biomechanical data can be derived from optical or translation sensors, and breathing rate data can be derived from respiration sensors. In a refinement, a computed asset can include one or more signals or readings from one or more non-animal data sources as one or more inputs in its one or more computations or calculations. In another refinement, a computed asset is comprised of a plurality of computed assets.

The term “predictive indicator” refers to a metric or other indicator (e.g., one or more colors, codes, numbers, values, graphs, charts, plots, readings, numerical representations, descriptions, text, physical responses, auditory responses, visual responses, kinesthetic responses) from which one or more forecasts, predictions, probabilities, possibilities, or recommendations related to one or more outcomes for one or more future events that includes one or more targeted individuals, or one or more groups of targeted individuals, can be calculated, computed, derived, extracted, extrapolated, simulated, created, modified, enhanced, estimated, evaluated, inferred, established, determined, deduced, observed, communicated, or actioned upon. In a refinement, a predictive indicator is a calculated computed asset derived from at least a portion of the animal data or its one or more derivatives. In another refinement, a predictive indicator includes one or more signals or readings from one or more non-animal data sources as one or more inputs in the one or more calculations, computations, derivations, extractions, extrapolations, simulations, creations, modifications, enhancements, estimations, evaluations, inferences, establishments, determinations, deductions, observations, or communications of its one or more forecasts, predictions, probabilities, possibilities, or recommendations. In yet another refinement, a predictive indicator is comprised of a plurality of predictive indicators.

The term “artificial data” refers to artificially-created data that is derived from or generated using, at least in part, real animal data or its one or more derivatives. It can be created by running one or more simulations utilizing one or more artificial intelligence techniques or statistical models, and can include one or more signals or readings from one or more non-animal data sources as one or more inputs. Artificial data also includes any artificially-created data that shares at least one biological function with a human or other animal (e.g., artificially-created vision data, artificially-created movement data). It is inclusive of “synthetic data,” which can be any production data applicable to a given situation that is not obtained by direct measurement. Synthetic data can be created by statistically modeling original data and then using those models to generate new data values that reproduce at least one of the original data's statistical properties. For the purposes of the presently disclosed and claimed subject matter, the terms “simulated data” and “synthetic data” are synonymous and used interchangeably with “artificial data,” and reference to any one of the terms should not be interpreted as limiting but rather as encompassing all possible meanings of all the terms.

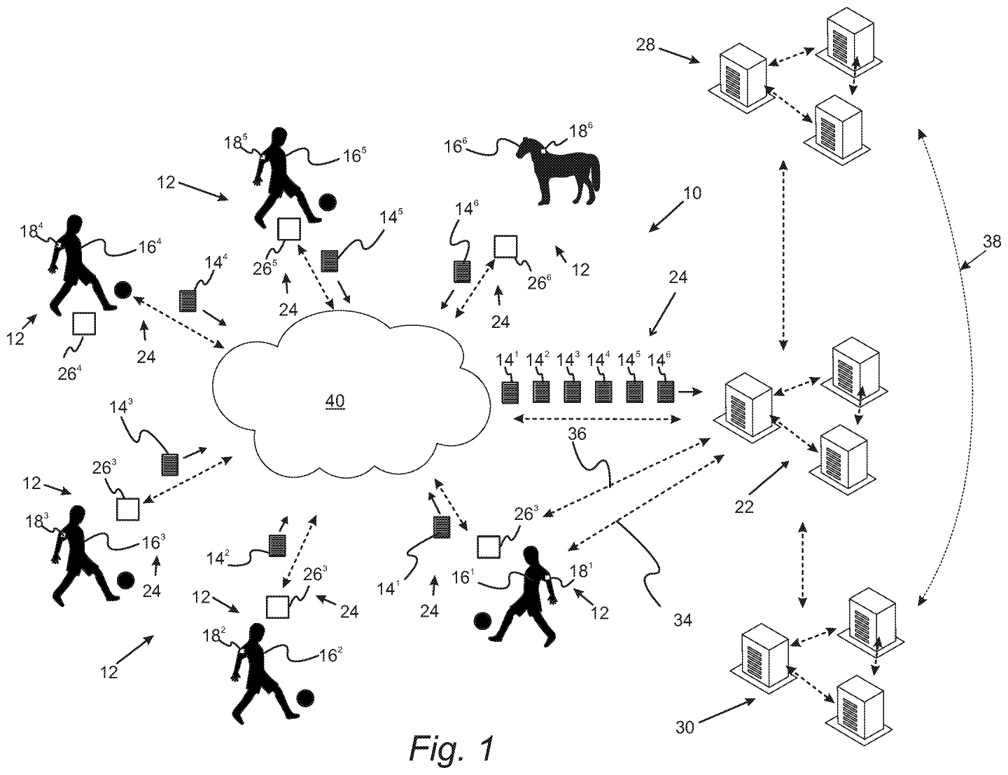

With reference to FIG. 1, a schematic of a system for providing animal data and predictive indicators thereof is provided. Speculation system 10 includes a source 12 of animal data 14ⁱ that can be transmitted electronically. Characteristically, source 12 of animal data includes one or more sensors 18ⁱ. Targeted individual or subject 16ⁱ is the subject from which corresponding animal data 14ⁱ is collected. Label i is merely an integer label from 1 to iₘₐₓ associated with each targeted individual where iₘₐₓ is the total number of individuals which can be 1 to several thousand or more. In this context, animal data refers to data related to a subject's body derived from sensors and in particular, biological sensors (biosensors). In many useful applications, the subject is a human (e.g., an athlete, a soldier, a hospital patient or remote telehealth patient, a participant in a fitness class, a video gamer) and the animal data is human data. Animal data can be derived from a targeted individual, multiple targeted individuals, a targeted group of multiple individuals, or multiple targeted groups of multiple individuals. The animal data can be obtained from a single source sensor on each targeted individual, or from multiple source sensors on each targeted individual. In some cases, a single source sensor can capture data from multiple individuals, a targeted group of multiple individuals, or multiple targeted groups of multiple individuals (e.g., an optical-based camera sensor that can locate and measure distance run for a target group of individuals). Each source sensor can provide a single type of animal data or multiple types of animal data. In a refinement, the one or more source sensors consist of at least one biosensor.

Biosensors collect biosignals which in the context of the present embodiment are any signals or properties in, or derived from, animals that can be continually or intermittently measured, monitored, observed, calculated, computed, inputted, or interpreted, including both electrical and non-electrical signals, measurements, and artificially-generated information. A biological sensor can gather biological data (e.g., including readings and signals) such as physiological, biometric, chemical, biomechanical, genetic, genomic, location or other biological data from one or more targeted individuals. For example, some biosensors may measure, or provide information that can be converted into or derived from, biological data such as eye tracking data (e.g., pupillary response, movement, EOG-related data), blood flow/volume data (e.g., PPG data, pulse transit time, pulse arrival time), biological fluid data (e.g., analysis derived from blood, urine, saliva, sweat, cerebrospinal fluid), body composition data (e.g., BMI, % body fat, protein/muscle), biochemical composition data, biochemical structure data, pulse data, oxygenation data (e.g., SpO2), core body temperature data, skin temperature data, galvanic skin response data, perspiration data (e.g., rate, composition), blood pressure data (e.g., systolic, diastolic, MAP), hydration data (e.g., fluid balance I/O), heart-based data (e.g., heart rate, average HR, HR range, heart rate variability, HRV time domain, HRV frequency domain, autonomic tone, ECG-related data including PR, QRS, QT, RR intervals), neurological-related data (e.g., EEG-related data), genetic-related data, genomic-related data, skeletal data, muscle data (e.g., EMG-related data including surface EMG, amplitude), respiratory data (e.g., respiratory rate, respiratory pattern, inspiration/expiration ratio, tidal volume, spirometry data), thoracic electrical bioimpedance data, or a combination thereof. Some biosensors may detect biological data such as biomechanical data which may include, for example, angular velocity, joint paths, gait description, step count, or position or accelerations in various directions from which a targeted subject's movements may be characterized. Some biosensors may gather biological data such as location and positional data (e.g., GPS, RFID-based data; posture data), facial recognition data, kinesthetic data (e.g., physical pressure captured from a sensor located at the bottom of a shoe), or auditory data related to the one or more targeted individuals. Some biological sensors are image or video-based and collect, provide and/or analyze video or other visual data (e.g., still or moving images, including video, MRIs, computed tomography scans, ultrasounds, X-rays) upon which biological data can be detected, measured, monitored, observed, extrapolated, calculated, or computed (e.g., biomechanical movements, location, a fracture based on an X-Ray, or stress or a disease based on video or image-based visual analysis of a subject). Some biosensors may derive information from biological fluids such as blood (e.g., venous, capillary), saliva, urine, sweat, and the like including triglyceride levels, red blood cell count, white blood cell count, adrenocorticotropic hormone levels, hematocrit levels, platelet count, ABO/Rh blood typing, blood urea nitrogen levels, calcium levels, carbon dioxide levels, chloride levels, creatinine levels, glucose levels, hemoglobin A1c levels, lactate levels, sodium levels, potassium levels, bilirubin levels, alkaline phosphatase (ALP) levels, alanine transaminase (ALT) levels, and aspartate aminotransferase (AST) levels, albumin levels, total protein levels, prostate-specific antigen (PSA) levels, microalbuminuria levels, immunoglobulin A levels, folate levels, cortisol levels, amylase levels, lipase levels, gastrin levels, bicarbonate levels, iron levels, magnesium levels, uric acid levels, folic acid levels, vitamin B-12 levels, and the like. In addition to biological data related to one or more targeted individuals, some biosensors may measure non-biological data conditions such as ambient temperature and humidity, elevation, and barometric pressure. In a refinement, one or more sensors provide biological data that include one or more calculations, computations, predictions, probabilities, possibilities, estimations, evaluations, inferences, determinations, deductions, observations, or forecasts that are derived, at least in part, from biosensor data. In another refinement, the one or more biosensors are capable of providing two or more types of data, at least one of which is biological data (e.g., heart rate data and VO2 data, muscle activity data and accelerometer data, VO2 data and elevation data).

The at least one sensor 18ⁱ and/or its one or more appendices can be affixed to, in contact with, or send one or more electronic communications in relation to or derived from, the subject including a subject's skin, eyeball, vital organ, muscle, hair, veins, biological fluid, blood vessels, tissue, or skeletal system, embedded in a subject, lodged or implanted in a subject, ingested by a subject, or integrated to comprise at least a portion of a subject. For example, a saliva sensor affixed to a tooth, a set of teeth, or an apparatus that is in contact with one or more teeth, a sensor that extracts DNA information derived from a subject's biological fluid or hair, a sensor that is wearable (e.g., on a human body), a sensor affixed to or implanted in the subject's brain that may detect brain signals from neurons, a sensor that is ingested by an individual to track one or more biological functions, a sensor attached to, or integrated with, a machine (e.g., robot) that shares at least one characteristic with an animal (e.g., a robotic arm with an ability to perform one or more tasks similar to that of a human; a robot with an ability to process information similar to that of a human), and the like. Advantageously, the machine itself may be comprised of one or more sensors, and may be classified as both a sensor and a subject. In a refinement, the one or more sensors 18ⁱ is integrated into or as part of, affixed to or embedded within, a textile, fabric, cloth, material, fixture, object, or apparatus that contacts or is in communication with a targeted individual either directly or via one or more intermediaries or interstitial items. Examples include a sensor attached to the skin via an adhesive, a sensor integrated into a watch or headset, a sensor integrated or embedded into a shirt or jersey, a sensor integrated into a steering wheel, a sensor integrated into a video game controller, a sensor integrated into a basketball that is in contact with the subject's hands, a sensor integrated into a hockey stick or a hockey puck that is in intermittent contact with an intermediary being held by the subject (e.g., hockey stick), a sensor integrated or embedded into the one or more handles or grips of a fitness machine (e.g., treadmill, bicycle, bench press), a sensor that is integrated within a robot (e.g., robotic arm) that is being controlled by the targeted individual, a sensor integrated or embedded into a shoe that may contact the targeted individual through the intermediary sock and/or adhesive tape wrapped around the targeted individual's ankle, and the like. In another refinement, one or more sensors may be interwoven into, embedded into, integrated with, or affixed to, a flooring or the ground (e.g., artificial turf grass, basketball floor, soccer field, a manufacturing/assembly-line floor), a seat/chair, helmet, a bed, or an object that is in contact with the subject either directly or via one or more intermediaries (e.g., a subject that is in contact with a sensor in a seat via a clothing interstitial). In another refinement, the sensor and/or its one or more appendices may be in contact with one or more particles or objects derived from the subject's body (e.g., tissue from an organ, hair from the subject) from which the one or more sensors derive or provide information that can be converted into biological data. In yet another refinement, one or more sensors may be optically-based (e.g., camera-based) and provide an output from which biological data can be detected, measured, monitored, observed, extracted, extrapolated, inferred, deducted, estimated, determined, calculated, or computed. In yet another refinement, one or more sensors may be light-based and use infrared technology (e.g., temperature sensor or heat sensor) to calculate the temperature of an individual or the relative heat of different parts of an individual.

In the variation depicted in FIG. 1, each individual 16ⁱ has at least one sensor 18ⁱ that gathers animal data 14ⁱ from the targeted individual 16ⁱ. Computing subsystem 22 receives and collects the animal data 14ⁱ through transmission subsystem 24. Transmission subsystem 24 enables the one or more source sensors 18ⁱ to transmit data wirelessly via one or more transmission (e.g., communication) protocols. Advantageously, sensor communication may occur in real-time or near real-time. In this context, near real-time means that the transmission is not purposely delayed except for necessary processing by the sensor and computing subsystem. Computing subsystem 22 is operable to receive the animal data or groups of animal data from a single targeted individual or multiple targeted individuals as raw or processed (e.g., manipulated) animal data. In a refinement, computing subsystem 22 is operable to receive a single type of animal data (e.g., heart rate data) and/or multiple types (e.g., including groups/data sets) of animal data (e.g., raw analog front end data, heart rate data, muscle activity data, accelerometer data, hydration data) from a single sensor and/or multiple sensors derived from a single targeted individual and/or multiple targeted individuals. In another refinement, transmission subsystem 24 includes computing device 26ⁱ which mediates the sending of animal data 14ⁱ to intermediate server 22, i.e., it collects the data and transmits it to computing subsystem 22. For example, computing device 26ⁱ can be a smartphone or a computer. However, computing device 26ⁱ can be any computing device. Typically, computing device 26ⁱ is local to the targeted individual or group of targeted individuals, although not a requirement for the present invention. In a further refinement, computing subsystem 22 communicates with the source 12 of animal data through cloud 40 or a local server (e.g., a localized or networked server/storage, localized storage device, distributed network of computing devices). Cloud 40 can be the internet, a public cloud, a private cloud utilized by the organization operating intermediate server 22 or other third party. Therefore, in this context, cloud 40 and/or the local server are part of transmission subsystem 24. In another refinement, transmission subsystem 24 includes direct communication links. Therefore, in this refinement computing subsystem 22 communicates directly with the source of animal data as shown by communication links 34 with sensor 18ⁱ or by communication link 36 with computing device 26ⁱ. In one refinement, the communication with sensor 18ⁱ can be via one or more designated transmission protocols or networks (e.g., wired, WIFI, BLE, Zigbee, NFC, cellular networks). In another refinement, the communication with sensor 18ⁱ can be via the sensor's native application or other data collection medium (e.g., cloud, server). In another refinement, communication with the one or more sensors may be via direct contact between a sensor (e.g., at the bottom of a shoe) and a receiving technology (e.g., integrated as part of the floor or ground).

Still referring to FIG. 1, computing subsystem 22 and/or one or more sensors 18ⁱ transform at least a portion of the animal data into at least one computed asset assigned to a selected targeted individual or a group of targeted individuals. Moreover, computing subsystem 22 and/or one or more sensors 18ⁱ are operable to transform the at least one computed asset into a predictive indicator. The computing subsystem 22 and/or one or more sensors 18ⁱ is further operable to provide the one or more outputs to one or more users. A user can be, for example, an end user of the one or more outputs via computing subsystem 22 such as a customer or acquirer of the data (e.g., a person or group of persons placing one or more wagers based on the one or more outputs in a field such as sports gambling, or a person or group of persons using the one or more outputs for their own health monitoring). In this regard, a user can be one or more persons, organizations, and the like. In a refinement, a user may be comprised of a plurality of users. A user can also be one or more systems or subsystems. A system can be one or more sets of one or more interrelated or interacting components which work together towards achieving one or more common goals or producing one or more desired outputs. The one or more components of a system can include one or more applications, frameworks, platforms or other subsystems, which may be integral to the system or separate from the system but part of a network or multiple networks linked with the system and operable to achieve the one or more common goals or produce the one or more desired outputs. For example, computing subsystem 22 may provide the one or more outputs to a user (system) such as a platform that provides insurance products based upon the one or more outputs, a telehealth application that provides real-time health statistics based upon the one or more outputs, or wagering system 28 or probability assessment system 30. In these examples, the one or more outputs being provided by computing subsystem 22 may be to one or more third party systems or subsystems, or one or more systems or subsystems directly or indirectly part of computing subsystem 22 (e.g., integral to computing subsystem 22, separate and operated by the one or more entities executing the computing subsystem 22). In a refinement, the one or more systems can be operated by the same entity operating computing subsystem 22 or by one or more different entities. In another refinement, the one or more systems are other systems not part of computing subsystem 22 but operated by the same entity operating the computing subsystem or one or more different entities.

In some variations, the transformation of at least a portion of the animal data into a computed asset, and the transformation of a computed asset into a predictive indicator, can occur via computing subsystem 22 or the one or more source sensors. Transformation can occur utilizing any animal data. For example, in the context of measuring a heart rate, a biological sensor can be configured to measure electric signals in the subject's body, transforming (e.g., converting) analog measurements to digital readings, and transmitting the digital readings. The computing subsystem can receive the digital readings and transform the digital readings into one or more heart rate values via one or more calculations based on overlapping segments of the digital readings by (i) identifying R-peaks within the overlapping segments, (ii) calculating a number of sample values based on times between adjacent R-peaks, (iii) discarding samples that are influenced by false peak detection or missed peak detection, and (iv) calculating an average, which may be weighted, of remaining sample values. The computing subsystem may determine that samples are influenced by false peak detection or missed peak detection in response to a sample value differing from a previous heart rate value by more than a first threshold. If a standard deviation of differences between samples is greater than a second threshold, the server may determine that samples are influenced by false peak detection or missed peak detection in response to the sample value differing from the previous heart rate value by more than a third threshold less than the first threshold. In a refinement, each step in a process that takes one or more actions upon the data can be considered a transformation for the purposes of the present invention. In this context, one or more or actions can include one or more calculations, computations, derivations, incorporations, simulations, extractions, extrapolations, modifications, enhancements, creations, estimations, deductions, inferences, determinations, processes, communications, and the like. In another refinement, one or more transformations occur utilizing one or more signals or readings from non-animal data.

In a refinement of one or more transformations related to measuring a heart rate, the at least one biological sensor is configured to measure electric signals in a subject's body, convert one or more analog measurements to one or more digital readings, and transmit the one or more digital readings. The computing subsystem is configured to receive the one or more digital readings and calculate heart rate based on one or more overlapping segments of the one or more digital readings by identifying R-peaks within the one or more overlapping segments, calculating one or more sample values based on times between adjacent R-peaks, discarding one or more samples that are influenced by false peak detection or missed peak detection, and calculating one or more averages of remaining sample values. The computing subsystem can be operable to communicate the one or more averages of the remaining sample values.

In another refinement of the one or more transformations related to measuring a heart rate, the at least one biological sensor is adapted for fixation to a subject's skin and configured to measure electric signals in the skin, convert analog measurements to digital readings, and transmit the digital readings. The computing system receives the digital readings and calculates the one or more heart rate values based on one or more overlapping segments of the digital readings by (i) identifying R-peaks within the one or more overlapping segments, (ii) calculating a number of sample values based on times between adjacent R-peaks, (iii) selecting samples within a first threshold of a previous heart rate value, and (iv) setting a current heart rate value to an average of the selected samples, which may be weighted. Each sample value may be proportional to a reciprocal of a time between adjacent R-peaks. The computing system may select samples within a second threshold of the previous heart rate value in response to a standard deviation of differences between consecutive samples being greater than a third threshold. The computing subsystem may set the current heart rate value equal to the previous heart rate value in response to the number of samples being less than a fourth threshold or in response to no samples being selected. The computing system can be operable to communicate the one or more current heart rate values to one or more users. The system may operate in real-time or near real-time wherein the computing system is operable to display each current heart rate value before a respective succeeding heart rate value is calculated and the computing system calculates each current heart rate value before the sensor completes measuring at least a portion of or all of the readings used to calculate the succeeding heart rate value. The computing system may compute an initial heart rate value by receiving a preliminary segment of the digital readings longer than the overlapping segments, identifying R-peaks within the preliminary segment, calculating sample values based on times between adjacent R-peaks, and calculating an average of the samples, which may be weighted.

In yet another refinement of one or more transformations related to measuring a heart rate, the at least one biological sensor is configured to measure one or more electric signals in a subject's body, transform (e.g., convert) analog measurements to one or more digital readings, and transmit the digital readings. The computing subsystem is configured to receive the one or more digital readings and transform (e.g., calculate) one or more heart rate values based on one or more overlapping segments of the one or more digital readings by identifying R-peaks within the one or more overlapping segments, calculating one or more sample values based on times between adjacent R-peaks, selecting one or more samples within a first threshold of a previous heart rate value, and setting a current heart rate value to an average of selected samples.

In still another refinement of one or more transformations related to measuring a heart rate, one or more readings are received by the computing subsystem from the at least one biological sensor, with the computing subsystem operable to process the one or more readings. For example, a first segment of readings is received by the computing subsystem from the one or more sensors. R-peaks within the first segment are then identified by the computing subsystem. Then, a first plurality of sample values is calculated by the computing subsystem based on times between adjacent R-peaks. For example, a constant may be divided by times between adjacent R-peaks. A first subset of the first plurality of sample values are selected including only sample values within a first threshold of a previous heart rate value. Then, a first updated heart rate value is calculated by the computing subsystem based on an average of the first subset of sample values. The first updated heart rate value can then be displayed by the computing subsystem. In later iterations, a second segment of the digital readings may be received by the computing subsystem from the one or more sensors. A third segment of digital readings may be formed by appending the second segment to the first segment. R-peaks within the third segment may then be identified. A second plurality of sample values may be calculated based on times between adjacent R-peaks. Then, a plurality of differences between consecutive samples may be calculated. In response to a standard deviation of the differences exceeding a second threshold, a second subset of the second plurality of sample values may be selected, including only sample values within a third threshold of the first updated heart rate value. A second updated heart rate value may then be calculated by the computing subsystem and displayed based on an average of the second subset of sample values, which may be weighted. An initial heart rate value may be calculated based on a preliminary segment of the digital readings.

In still another refinement of one or more transformations related to measuring a heart rate, transformation can occur when addressing issues related to signal quality. In cases where the raw data has an extremely low signal-to-noise ratio, additional pre-filter logic may be applied to transform the data prior to calculating a heart rate value. The pre-filter process detects any outlier values and replaces the one or more outlier values, using a look-ahead approach, with values that align in the time series of generated values and fit within a preestablished threshold/range. These generated values that fit within a preestablished threshold/range can be passed along through the system for its computation of the one or more heart rate values.

In yet another refinement of one or more transformations related to measuring a heart rate, transformation can occur when detecting and replacing one or more outlier values generated from one or more biological sensors. The computing subsystem can be operable to receive one or more values generated directly or indirectly by the one or more biological sensors. One or more statistical tests can be applied by the computing subsystem to determine an acceptable upper and/or lower bound for each value. A backward filling method can be used to replace the one or more outlier values with a next available value that falls within an acceptable range established in a current window of samples.

Additional details related to a system for measuring a heart rate and other biological data are disclosed in U.S. patent application Ser. No. 16/246,923 filed Jan. 14, 2019 and U.S. Pat. No. PCT/US20/13461 filed Jan. 14, 2020; the entire disclosures of which are hereby incorporated by reference. The present invention is not limited to the methods or systems used to transform animal data and/or its one or more derivatives, nor is the present invention limited to the type of data being transformed.

The one or more outputs provided by speculation system 10 can include one or more predictive indicators, computed assets, animal data (including signals and readings), its one or more derivatives, and/or a combination thereof. In this context, “provided” includes “sent”, “made available,” and “granted access to.” For example, computing subsystem can send the one or more outputs to another one or more systems or subsystems including platforms and applications (e.g., wagering application, health/telehealth application, fitness application, insurance application, prediction application, rehabilitation application), or grant access to the one or more outputs should the other system or subsystem access the data via one or more mechanisms (e.g., access via cloud 40). Computing subsystem 22 is operable to use at least a portion of the one or more outputs from computing subsystem 22 either directly or indirectly for the following applications: (1) as a market upon which one or more wagers are placed or accepted; (2) to accept one or more wagers; (3) to create, enhance, modify, acquire, offer, or distribute one or more products; (4) to evaluate, calculate, derive, modify, enhance, or communicate one or more predictions, probabilities, or possibilities; (5) to formulate one or more strategies; (6) to take one or more actions; (7) to mitigate or prevent one or more risks; (8) as one or more signals or readings (e.g., including sets of signals of readings) utilized in one or more simulations, computations, or analyses; (9) as part of one or more simulations, an output of which directly or indirectly engages with one or more users; (10) to recommend one or more actions; (11) as one or more core components or supplements to one or more mediums of consumption; (12) in one or more promotions; or (13) a combination thereof. In this context, a combination can include two or more, as well as all possible iterations. Furthermore, indirect use can include any derivative of the one or more outputs, or non-direct application of the one or more outputs. For example, if a probability is created for subject X based on predictive indicator X and a probability is created for subject Y based on predictive indicator Y, a probability for Group Z (comprised of Subjects X and Y) may be created without direct use of predictive indicators X and Y. Indirect use of the one or more outputs can also include one or more actions that are not directly derived from the data. For example, indirect use can include observation of a user's interaction with the data, from which computing subsystem 22 or the wagering system 28 or the probability assessment system 30 may dynamically create, enhance, or modify a wagering market or odds, a product that is acquired or consumed, a strategy, a prediction, a recommendation, and the like based upon the user interaction with the data rather than based on the data itself. Lastly, one or more uses may be interconnected or interrelated. For example, an action may also mitigate a risk, creation of a probability may enable formulation of a strategy, creation of a product may be utilized in a promotion, a simulation output may provide the basis for a prediction or recommendation, and the like.

In a variation with respect to application (1), a market can be a specific type or category of bet on a particular event. A market can be for any event. Oftentimes, organizations that accept one or more bets offer a plurality of betting markets on each event, with odds listed for each market. Specific types or categories can include a proposition bet, spread bet, a line bet, a future bet, a parlay bet, a round-robin bet, a handicap bet, an over/under bet, a full cover bet, or a teaser bet.

In a variation with respect to application (2), acceptance of a wager can be, for example, acceptance of a bet by a wagering system utilizing the one or more outputs (e.g., a bet type derived from the predictive indicator), acceptance by an insurance system (e.g., insurance provider) of a payment from an individual that is correlated with a taken risk by the insurance provider based upon the one or more outputs (e.g., the insurance policy provided to an individual, which may or may not cost the company more money, based on the likelihood of the individual experiencing any given biological event forecasted by the predictive indicator), and the like.

In a variation with respect to application (3), one or more products can be one or more goods or services that are designed to be sold or distributed. A product can be any product in any industry or vertical that can be created, modified, enhanced, offered, or distributed, so long as the product uses at least a portion of the one or more outputs either directly or indirectly. It is inclusive of one or more outputs leading to (or resulting in) the creation of a product. For example, a product can be the one or more outputs itself (e.g., the predictive indicator), a market to bet on, an insurance offering, a health application that displays the one or more outputs, a suite of algorithms designed to provide a particular insight related to a subject, a sports betting application, a consumer product that utilizes the one or more outputs (e.g., beverages, foods), and the like. For clarification purposes, “enhance” can include “to be part of” a product should the enhancement add value. In addition, and in many cases, “create” can be inclusive of “derive” and vice versa. Similarly, “create” can be inclusive of “generate” and vice versa. Furthermore, “modify” can be inclusive of “revise”, “amend”, “adjust”, “change”, and “refine.” Lastly, an “acquirer” of a product could be, for example, a consumer, an organization, another system, any other end point that could consume the product, and the like.

In a variation with respect to application (4), the one or more predictions, probabilities, or possibilities can be related to a future outcome or occurrence, with one or more predictions, probabilities, or possibilities connected. For example, a probability may be calculated to determine the likelihood of any given athlete elevating his heart rate over 200 beats per minute in any given basketball game utilizing various types of data including the athlete's current heart rate, average heart rate, max heart rate, historical heart rate for similar conditions, biological fluid levels, sEMG data, the number of minutes on the court, total distance run, and the like. Utilizing this probability, another probability may be calculated to determine the likelihood that the athlete will make baskets outside of 25 feet at a percentage exceeding 50%. In addition, “communication” can include visualization of the one or more predictions, probabilities, or possibilities (e.g., displaying a probability via an application, displaying an output-based probability for another individual within an AR or VR system), verbal communication of one or more predictions, probabilities, or possibilities (e.g., a voice-activated virtual assistant that informs an individual of the likelihood any event can occur based on their one or more outputs, or that an event will happen. An example could be the likelihood of having low blood sugar if a certain action is not taken, the likelihood of having a stroke in the next 120 days based on the collected biological data, or that a biological-related event will occur based upon the one or more outputs). Lastly, modification of a prediction, probability, or possibility can include revising a previously determined prediction, probability, or possibility for an event.

In a variation with respect to application (5), a strategy can include any strategy that uses the one or more outputs. A strategy can be a plan of action to determine, for example, whether or not to insure an individual, whether or not to place a bet, whether or not to take a specific action, and the like.

In a variation with respect to application (6), an action can be any action that is directly or indirectly related to at least a portion of the one or more outputs. An action includes an action that is derived from (or results from) the one or more outputs. It can be, for example, an action to insure someone (e.g., a person's chances of having a heart attack in the next 24 months is X, so their premium will be Y), an action related to an individual's biology (e.g., a passenger in a car has an output reading that triggers a self-driving car to drive to the nearest hospital), an action to place a wager (e.g., the athlete's energy level is at X percent, therefore a user places a bet), an action to take a specific action (e.g., a system communicating an action to take a specific action such as “place a bet,” “run for 20 minutes today,” “eat X number of calories today”), an action to take no action at all, and the like.

In a variation with respect to application (7), mitigation or prevention of risk can include any action, non-action, strategy, recommendation, and the like related to reducing or preventing risk. It can also include taking additional risk.

In a variation with respect to application (8), a signal or reading can include any form and any format of information (e.g., including as one or more data sets).

In a variation with respect to application (9), a simulation includes both the production of one or more computer models, as well as imitation of one or more situations or processes. Simulations have a wide range of engagement uses, including simulations that are utilized to generate the one or more outputs, which any use of the outputs can be considered either direct or indirect engagement, as well as inclusion of the one or more outputs within one or more simulations, which may engage one or more users (e.g., a video game, an AR/VR system).

In a variation with respect to application (10), to recommend one or more actions includes both a recommendation that is inferred by the one or more outputs (e.g., a predictive indicator that provides a probability of an occurrence happening may infer an action to be taken) as well as a recommendation directly stated based on the one or more outputs (e.g., a recommendation that an action be taken based on a predictive indicator derived from a probability of an occurrence happening). In a refinement, a recommendation may be comprised of a plurality of recommendations.

In a variation with respect to application (11), the one or more mediums of user consumption can be any medium where a user can directly or indirectly consume the one or more outputs. A medium can include, for example, a health monitoring application that communicates a heart status check via the one or more outputs, a remote rehabilitation or telehealth platform that communicates the one or more outputs to the platform during an activity (e.g., remote exercise) while enabling the remote medical professional or rehabilitation specialist to see the patient via an integrated video display, an insurance application that communicates an insurance adjustment based at least in part on the animal data, a sports wagering platform, and the like. It can also include a media broadcast that incorporates the one or more outputs, a sports streaming content platform (e.g., video platform) that integrates the one or more outputs as a supplement to the live sports event being watched (e.g., enabling a user to place a wager while watching the live content), and the like.

In a variation with respect to application (12), the one or more promotions can be any promotion that provides support in furtherance of the acceptance and acquisition (e.g., sale) of one or more products. This includes one or more advertisements, an offer that uses the one or more outputs (e.g., an offer to obtain insurance with the potential of lowering a premium by providing the one or more outputs), a discounting mechanism that uses the one or more outputs, and the like.

In a variation, computing subsystem 22 is operable to provide the one or more data outputs to one or more systems (e.g., wagering system 28, probability assessment system 30, other systems), with the one or more systems operable to utilize at least a portion of the one or more outputs either directly or indirectly: (1) as a market upon which one or more wagers are placed or accepted; (2) to accept one or more wagers; (3) to create, enhance, modify, acquire, offer, or distribute one or more products; (4) to evaluate, calculate, derive, modify, enhance, or communicate one or more predictions, probabilities, or possibilities; (5) to formulate one or more strategies; (6) to take one or more actions; (7) to mitigate or prevent one or more risks; (8) as one or more signals or readings utilized in one or more simulations, computations, or analyses; (9) as part of one or more simulations, an output of which directly or indirectly engages with one or more users; (10) to recommend one or more actions; (11) as one or more core components or supplements to one or more mediums of consumption; (12) in one or more promotions; or (13) a combination thereof.

In another variation, the one or more outputs are dynamically created, modified, or enhanced by computing subsystem 22, with at least a portion of the dynamically created, modified, or enhanced one or more outputs utilized either directly or indirectly: (1) as a market upon which one or more wagers are placed or accepted; (2) to create, modify, enhance, acquire, offer, or distribute one or more products; (3) to evaluate, calculate, derive, modify, enhance, or communicate one or more predictions, probabilities, or possibilities; (4) to formulate one or more strategies; (5) to take one or more actions; (6) to mitigate or prevent one or more risks; (7) as one or more signals or readings utilized in one or more simulations, computations, or analyses; (8) as part of one or more simulations, an output of which directly or indirectly engages with one or more users; (9) to recommend one or more actions; (10) as one or more core components or supplements to one or more mediums of consumption; (11) in one or more promotions; or (12) a combination thereof. In a refinement, dynamic creation, modification, or enhancement may occur on another system (e.g., a system within a network of related systems, 3^{rd} party system).

In another variation, the one or more direct or indirect uses by the computing subsystem are dynamic, at least in part, and based upon one or more user interactions with the one or more outputs. For example, computing subsystem 22 may be operable to dynamically create, enhance, or modify at least one of: a wagering market or odds, a product that is acquired or consumed, an evaluation or calculation of a probability, a strategy, a prediction, a recommendation, or an action to mitigate or prevent risk based upon at least a portion of the one or more outputs from computing subsystem 22. Such creations, enhancements, or modifications may result from one or more direct or indirect observations of user engagement with data collected by computing subsystem 22, or as new data is collected by the system. Dynamic in this case refers to not being static with an ability to change based on or more factors or inputs. Such use cases can include the creation or enhancement of, or modification to, products in areas like sports betting (e.g., any type of wager or market including a proposition bet, a spread bet, a parlay, a future, a line bet, a round-robin, a teaser), non-sports betting products (e.g., platforms that utilize animal data-based predictive indicators such as a health monitoring application, telehealth application, fitness application, insurance application, rehabilitation application), strategies based upon user interaction with data, recommendations based on collected data, and the like. For example, if an individual is purchasing data related to heart rate for Team X, a proposition bet may be created and offered to the individual related to heart rate for Team X. In another example, if a user of data is viewing their own heart health statistics (e.g., ECG readings) coupled with other types of animal data (e.g., stress level, fatigue) via a health application, the computing subsystem may dynamically create or adjust its recommendation product based on the user's interaction with their health stats (e.g., based on irregular ECG patterns and extremely high stress levels, the product may communicate: “User has a 7% chance of having a heart attack in the next 30 days. Go see a doctor” based upon the system's analysis of the previously collected data). Such one or more functionalities can also be exhibited by the wagering system 28 and/or the probability assessment system 30.

In yet another variation, the speculation system 10 interacts with a wagering system 28 or probability assessment system 30. Wagering system 28 can be set up, for example, to receive one or more wagers from one or more individuals, with the computing subsystem 22 sending data (e.g., animal data and/or its one or more derivatives) to the wagering system. The wagering system typically includes one or more computing devices executing a wagering application. Similarly, the probability assessment system 30 can be set up, for example, to evaluate or calculate one or more probabilities, make one or more predictions, mitigate or prevent one or more risks, or create, enhance, or modify one or more products for acquisition or consumption, with the computing subsystem 22 sending data (e.g., animal data and/or its one or more derivatives) to the probability assessment system. The wagering system or the probability assessment system can be operated by the entity in control of the speculation system or by a third party. In a refinement, the wagering system 28 or probability assessment system 30, or a combination thereof, are part of computing subsystem 22. Characteristically, the computing subsystem 22 is operable to provide the same or substantially similar one or more data outputs to a plurality of users (which can include, for example, multiple systems or end users such as bettors). Advantageously, the providing of data to a plurality of users can occur concurrently. For example, the computing subsystem may provide the same “energy level” output for Athlete X to multiple systems (e.g., broadcast partners) but one of the outputs may include a different graphics package or include different metadata or formatting (e.g., time stamps displayed in different ways). In another example, the computing subsystem may provide the heart rate output to multiple users (e.g., multiple systems) from which a variety of products can be created. In yet another example, the computing subsystem may provide a wagering opportunity for the heart rate output for Athlete X but may display or communicate it to the one or more users (e.g., in this case, the one or more bettors) in multiple ways (e.g., a beats per minute number display to bettor A, and a beats per minute vibration notification alert sent to a smart watch for bettor B, a beats per minute number verbally communicated by a virtual assistant to bettor C). In a variation, the bet type may be based on the same heart rate output of Athlete X but may be productized in different ways to accommodate the one or more bettors and their preferred wagering products (e.g., providing a wager to a bettor for heart rate as a number vs heart rate as a color, which is based directly on the number). In a refinement, the one or more outputs of computing subsystem 22 are synchronized with one or more types of non-animal data and/or one or more mediums of consumption. For example, the one or more outputs may be synchronized with media content which can include video content (e.g., data may be synchronized with one or more live streams of a sporting event to offer an ability to place one or more wagers while watching a sporting event; the data may be synchronized with the streaming video of a patient during a real-time telehealth or remote monitoring or rehabilitation session; the data may be synchronized with visual content derived from and utilized within smart glasses or AR/VR systems), audio content, additional data readings (e.g., statistics in sports like points won/lost, matches won/lost, points scored, assists, goals, shot percentage, and the like), a simulation game (e.g., video game), and the like. In another refinement, computing subsystem 22 or wagering system 28 or probability assessment system 30 are operable to create one or more betting products from animal data.

In addition, the one or more wagering systems and probability assessment systems may share one or more functionalities and/or characteristics (e.g., both types of systems may be programmed to evaluate one or more probabilities, formulate one or more strategies, inform one or more users to take one or more actions, provide one or more recommendations, mitigate one or more risks, create or modify one or more products). Similarly, one or more wagering systems may take on one or more functionalities or characteristics of a probability assessment system and vice versa. In a refinement, the wagering system and the probability assessment system may operate together (e.g., within the same one or more networks or performing different tasks to solve for the same use case) to provide one or more different offerings based on the same data for the same or similar use case. In some cases, one or more wagering systems may communicate directly 38 with one or more probability assessment systems and vice versa. For example, upon receiving data from computing subsystem 22, a probability assessment system may create a product that provides one or more odds of any given outcome occurring (e.g., in sports betting, insurance, healthcare), and a wagering system may accept the wager based on the one or more odds (e.g., a sports betting platform that creates and accepts a wager with odds created based on a predictive indicator by a third party analytics company; an insurance company that creates and accepts the risk to insure someone via an adjusted insurance premium based on a predictive indicator created by a third-party insurance analytics company; a telehealth or remote health monitoring company that accepts risk of providing a digital services product to a patient based on a predictive indicator provided by a third party).

As set forth above, the predictive indicator provides a plurality of opportunities for value creation, including as a basis for new wagering markets and products, as well as establishing predictions and related determinations (e.g., probabilities, possibilities) associated with future occurrences. The predictive indicator can be derived in a variety of ways; for example, by utilizing one or more statistical models, by one or more artificial intelligence techniques (e.g., machine learning, deep learning techniques), or by one or more calculations or computations. For example, by utilizing one or more machine learning methods, the system can analyze previously-collected and current data sets to create, modify, or enhance one or more predictions. Given that machine learning-based systems are set up to learn from collected data rather than require explicit programmed instructions, its ability to search for and recognize patterns that may be hidden within one or more data sets enable machine learning-based systems to uncover insights from collected data that allow for predictions to be made. Advantageously, because machine learning-based systems use data to learn, it oftentimes takes an iterative approach to improve model prediction and accuracy as new data enters the system, as well as improvements derived from feedback provided from previous computations made by the system (which also enables production of reliable and repeatable results).

In addition, the predictive indicator can be represented in n number of ways. For example, the predictive indicator may be represented as a percentage (e.g., there is a 75% chance subject X will have a heart attack in the next n years), as text or a statement (e.g., a recommended action based on the predictive indicator such as “drink water in the next n minutes or subject X will be dehydrated”; a statement such as “subject X will have an experience y medical condition in the next n days”), as a physical response (e.g., vibration via a watch that is programmed to alert a user to place a bet based on the predictive indicator), and the like. The predictive indicator can be derived from a single animal data type or from multiple animal data types. It can be derived from any signals, readings, or derivatives of animal data, or any portion thereof. In a refinement, the predictive indicator is a calculated computed asset from at least a portion of the animal data or a composite calculated from two or more signals or readings from one or more source sensors. For example, one or more physiological metrics may be predictive of targeted individual's fatigue level thereby predicting the success of such individual to performing certain tasks at that particular time (e.g., in sports, make a free throw or kick a field goal; as a pilot, drive or fly safely to a destination; as a surgeon, perform a surgery). In this situation, the predictive indicator can be used, for example, to determine whether or not to place a bet, to determine the probability of an outcome occurring for an event, to revise previously determined probability for an event, or formulate a strategy upon which a market is created for individuals to place a wager on or upon which an action is taken. Therefore, the user may be an organization evaluating a risk or accepting a risk for financial gain (e.g., bookmaker, insurance company), analytics company, a sports team analyzing a player's performance, a person placing a bet, or a company that creates betting products. In a variation, predictive indicator is calculated from a computed asset that includes biological data selected from the group consisting of: facial recognition data, eye tracking data, blood flow data, blood volume data, blood pressure data, biological fluid data, body composition data, biochemical composition data, biochemical structure data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, thoracic electrical bioimpedance data, or a combination thereof. In another refinement, the predictive indicator includes one or more signals or readings from non-animal data. The one or more non-animal signals or readings can include, for example, ambient temperature data, humidity data, barometric pressure data, elevation data, wind velocity, nutrition data, family history data, psychological data, non-animal statistical data (e.g., examples in the context of sport include points, rebounds, assists, touchdowns, shots, goals, turnovers, yards passed, yards run, win/loss, winning %, and head-to-head information), other historical data, and the like.

In another refinement, at least a portion of the predictive indicator is used either directly or indirectly: (1) as a market upon which one or more wagers are placed or accepted; (2) to create, modify, enhance, acquire, offer, or distribute one or more products; (3) to evaluate, calculate, derive, modify, enhance, or communicate one or more predictions, probabilities, or possibilities; (4) to formulate one or more strategies; (5) to take one or more actions; (6) to mitigate or prevent one or more risks; (7) as one or more readings utilized in one or more simulations, computations, or analyses; (8) as part of one or more simulations, an output of which directly or indirectly engages with one or more users; (9) to recommend one or more actions; (10) as one or more core components or supplements to one or more mediums of consumption; (11) in one or more promotions; or (12) a combination thereof. For illustration purposes, the one or more actions in this context could include the acceptance of one or more wagers. In a healthcare scenario, a user (e.g., a patient) may accept to pay for the cost of a medication or prescription written by a medical professional (e.g., doctor), with the prescription or medication being prescribed based upon the predictive indicator (e.g., the predictive indicator may indicate that there may be a n percent chance of the patient experiencing a medical condition; therefore, the doctor prescribes pill x to reduce the likelihood of the medical event based upon the predictive indicator). The act of the patient accepting pill x—which was prescribed based at least in part on the predictive indicator as well as accepting the risk (e.g., the cost, potential health-related issues to pill x) in exchange for the benefit of taking the medication or prescription to improve their wellness can be a wager that is accepted and actioned upon by a patient/user.

In another refinement, the predictive indicator can include a plurality of predictive indictors. For example, a predictive indicator may provide multiple predictive assessments within a single indicator (e.g., an indicator that states the probability of X occurring within n months or the probability of Y occurring within n+3 months). In another refinement, one or more predictive indicators can be derived from or related to a targeted individual, multiple targeted individuals, a targeted group comprised of multiple targeted individuals, and/or multiple targeted groups comprised of multiple targeted individuals. This includes being applicable to, associated with, assigned to, and the like. For example, the one or more predictive indicators can be attributed to a targeted individual, multiple targeted individuals with each individual having their own one or more predictive indicators, a target group comprised of targeted individuals with the group having its own one or more predictive indicators and/or the individuals having their own or more predictive indicators, or multiple groups comprised of multiple targeted individuals, with the plurality of target groups having their own one or more predictive indicators and/or each target group within the multiple target groups having their own one or more predictive indicators and/or the targeted individuals within each target group of the plurality of groups having their own one or more predictive indicators. For example, in the context of a basketball team, Player A is guard for basketball ball Team B in League C. Player A could have a predictive indicator related to their performance, Team B could have a predictive indicator related to team performance derived, at least in part, from members of its team (including Player A), and League C could have a predictive indicator for all guards in its league which would be derived, at least in part, from all guards in its league (which would include Player A). In another refinement, one or more targeted individuals or target groups of targeted individuals include one or more anonymized individuals.

In yet another refinement, the predictive indicator includes at least a portion of biological data derived from one or more source sensors, and may provide a prediction regarding the targeted individual's health status, which could include feedback regarding life expectancy, risks associated with a medical treatment (e.g., surgery, drug treatment, etc.), or general wellness indicators including stress or energy level. Users of such information include aviation companies, medical facilities (e.g., hospitals), pharmaceutical companies, automotive companies, transportation companies, rehabilitation facilities, military organizations, sports organizations, local municipality groups (e.g., police), oil & gas companies, construction companies, healthcare companies, financial groups, insurance companies, corporate wellness, other technology companies, individuals, and the like. For example, an airline or transportation company can use the predictive indicator to monitor and predict pilot or driver fatigue. Insurance companies can apply the predictive indicator to adjust premiums for individuals based on the animal data collected and analyzed. A financial trading Firm can apply the predictive indicator to predict a person's stress levels based upon trade size and volume, which may impact decision-making abilities. A retirement facility or nursing home can apply the predictive indicator to determine future care needs that are expected for any given patient and therefore could adjust the fee required to provide care for the individual. A telehealth or remote health monitoring company can apply the predictive indicator to determine the likelihood of any given health outcome, and provide feedback to the patient as well as one or more recommended actions (e.g., take a prescribed medication; take a specific action to prevent a risk). A home gym equipment or fitness provider can use the predictive indicator to determine the future outcome of any given person's workout with the goal of providing a recommendation related to the workout (e.g., your body will fatigue in n seconds if you continue to run on the treadmill at z mph. You will need y minutes/seconds of recovery time before the next exercise). A sports bettor could apply the predictive indicator to assess one or more individuals' present or future biological state, which may influence their desire to place a bet. In another refinement, the predictive indicator may be used as an indicator to enable one or more actions to occur. For example, a taxi company may implement a system that utilizes the predictive indicator to certify that an individual is able to drive safely, which would lead to the individual driving a vehicle. A military organization may use the predictive indicator to determine the “readiness” of a solider for battle or other tasks, leading to an action taken upon or by a solider. A rehabilitation platform may utilize the predictive indicator to predict how well a subject may recover from any given injury, as well as to predict what exercise (or exercises) and/or rehabilitation techniques may be most effective for the subject to ensure the highest probability for recovery. An airline or union may use the predictive indicator to ensure a pilot is ready to fly on any given day, or extend the retirement age of certain pilots based upon their physiological characteristics or other collected animal data. In this example, the question may be whether to allow any given n-year old pilot (e.g., 65 years old) whose data has been collected by the system an ability to continue to fly past a certain age or while exhibiting specific biological characteristics which may include physiological, biomechanical, and neurological characteristics. More specifically, it may be in the airline's best interest to determine the biological “fitness” of the pilot and predict future biological fitness rather than mandating a work stoppage (e.g., mandatory retirement) due to an indicator such as a person's age, as the pilot's experience could lead to an overall safer flying experience and/or enable more routes to be flown to increase business. Therefore, the system may use one or more techniques (e.g., statistical models, run one or more simulations via one or more artificial intelligence techniques) for any given pilot on collected animal data (e.g., heart/ECG data, age, other data including weight, habits, medical history) to generate one or more predictive indicators (e.g., a predictive data set to see the pilot's heart activity from future ages 66-80 to determine future biological “fitness” as well as future “fitness for flying” over a defined period of time, from which a recommendation can be derived). In a refinement, computing subsystem 22 or the one or more wagering systems are operable to create one or more wagering opportunities from the predictive indicator.

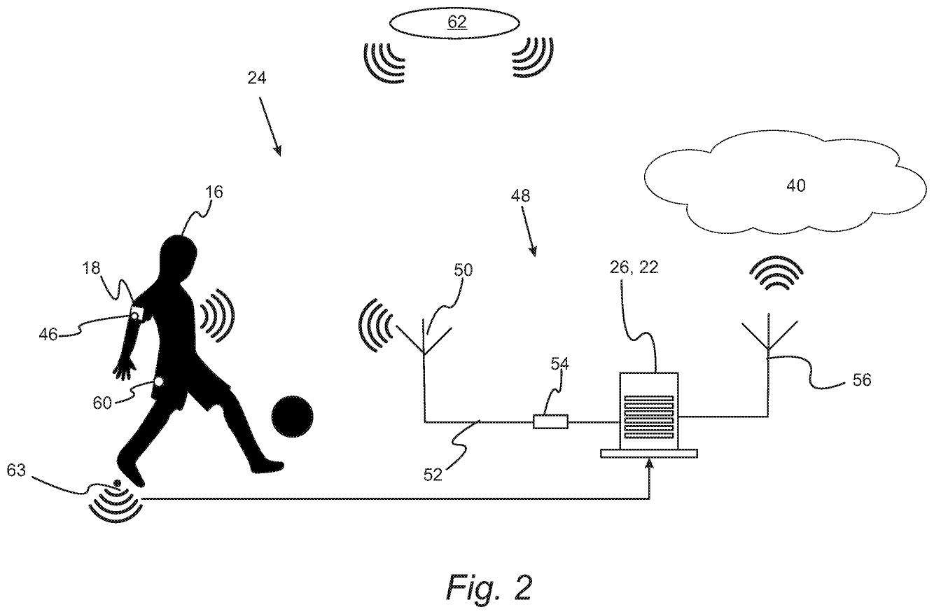

As set forth above, speculation system 10 includes transmission subsystem 24. Typically, transmission subsystem 24 includes a transmitter and a receiver, or a combination thereof (e.g., transceiver). Transmission subsystem 24 can include one or more receivers, transmitters and/or transceivers having a single antenna or multiple antennas (e.g., which may be configured as part of a mesh network). The transmission subsystem and/or its one or more components may be housed within the computing subsystem or may be external to the computing subsystem (e.g., a dongle connected to the computing device which is comprised of one or more hardware and/or software components that facilitates wireless communication and is part of the transmission subsystem). In a refinement, the transmission subsystem and/or one or more of its components are integral to, or comprised within, the one or more sensors. FIG. 2 depicts computing device 26 (or computing subsystem 22) receiving a signal from sensor 18 attached to targeted individual 16. Sensor 18 includes an integral transmitter, receiver, or transceiver 46. Advantageously, the transmission subsystem enables the one or more source sensors to transmit data wirelessly for real-time or near real-time communication. In addition, the transmission subsystem can communicate with the one or more source sensors utilizing one or more transmission protocols. The present invention is not limited by the technologies that sensors 18 use to transmit and/or receive signals. Currently, such transmission technologies and infrastructure include, but are not limited to, Bluetooth, Bluetooth Low Energy, Zigbee, Ant+, NFC, WIFI, cellular networks, and the like. Receiver 48 receives the signal from transmitter 46.

In the depicted variation, receiver 48 includes antenna and/or dongle (e.g., Bluetooth transceiver) 50 and computing device 26 (or computing subsystem 22). In a refinement, antenna/dongle 50 can be located at a far distance from computing device 26 (e.g., 100 feet, 1000 feet, or more). Therefore, connection line 52 may include a converter 54 (e.g., USB to Ethernet if required) that allows longer lines to run if required. Finally, adapter 56 is used by computing device 26 to transmit the animal data to the computing subsystem 22 of FIG. 1 via cloud 40 or local server. Cloud 40 can be the internet, a public cloud, or a cloud owned by the company operating the speculation system or third-party.

In many cases, the communication distance between a sensor and a receiver of the sensor signal can be elongated by the transmission subsystem for real-time or near real-time communication, thereby extending a range limitation of the one or more sensors and their corresponding one or more transmission protocols. In a refinement, the computing subsystem synchronizes communication and real-time or near real-time streaming for the one or more sensors that are communicating with computing subsystem 22. Advantageously, the transmission subsystem enables real-time or near real-time streaming in environments where potential radio frequency (RF) interference occurs. In a refinement, the computing subsystem 22 sends at least a portion of the animal data to another location (e.g., predetermined location within the system or another system) or stores the animal data for later use. In a variation, the system may provide a real-time or near real-time backup mechanism for incoming data from the one or more source sensors with minimal effect on the real-time or near real-time transmission.

In another refinement, the one or more transmission subsystems, or the one or more components of the transmission subsystem such as an antenna and/or dongle, may be wearable and may be affixed to, in contact with, or integrated with, the subject either directly or via one or more intermediaries (e.g., clothing). The transmission subsystems, or components of the transmission subsystem, may also be mobile or personal to the one or more individuals. In another refinement, transmission subsystem 24 includes an on or in-body transceiver 60 (“on-body transceiver”) that optionally acts as another sensor or is optionally integrated within a biological sensor. On-body transceiver 60 is operable to communicate with the one or more sensors 18 on a target subject or across one or more target subjects, and may itself track one or more types of biological data (e.g., positional or location data). In a refinement, the on-body transceiver is affixed to, integrated with, or in contact with, a subject's skin, hair, vital organ, muscle, skeletal system, eyeball, clothing, object, or other apparatus on a subject. Advantageously, the on-body transceiver collects the one or more data streams in real-time or near real-time from one or more sensors on a subject's body, communicating with each sensor using a transmission protocol of that particular sensor. The on-body transceiver may also act as a data collection hub. In a refinement, the on-body transceiver can minimize transmission overhead while enhancing the transmission capabilities (e.g., increase speed, reducing latency). In another refinement, the on-body transceiver can include logic that enables the on-body transceiver to perform at least one action on the animal data from the group consisting of: collecting, normalizing, time stamping, aggregating, tagging, storing, manipulating, denoising, productizing, enhancing, organizing, visualizing, analyzing, summarizing, replicating, synthesizing, anonymizing, synchronizing, or distributing the animal data. Characteristically, the on-body transceiver may be operable to send any collected and selected data (e.g., animal data, computed assets, predictive indicators, any derivatives, and the like) to n number of end points in real-time or near real-time, while enabling any data not selected to be stored on the transceiver for download at a later time. In addition, its summary capabilities enables data that may, for example, be sampled at high frequency rates (e.g., 250-1000 hz) to be summarized and sent in summarized form (e.g., the data processed and/or summarized at 1 hz) to accommodate any number of use cases or constraints (e.g., limited bandwidth).

In another variation, transmission subsystem 24 includes an aerial transceiver 62 for continuous streaming and/or intermittent communication from the one or more sensors located on one or more target subjects or objects. Examples of aerial transceiver 62 include, but are not limited to, one or more communications satellites or unmanned aerial vehicles with attached transceivers (e.g., high-altitude pseudo satellites, drones). Additional details of unmanned aerial vehicle-based data collection and distribution systems are disclosed in U.S. patent Ser. No. 16/517,012 filed Jul. 19, 2019; the entire disclosure of which is hereby incorporated by reference. In another variation, transmission subsystem 24 includes a transceiver 63 embedded or integrated as part of a floor or ground (including a field), with transmission occurring via direct contact with a surface (e.g., in the event the sensor is located on or near the bottom of the shoe).

In a variation, computing subsystem 22 synchronizes, time-stamps, and tags the animal data with information (e.g., characteristics) related to the one or more targeted individuals from which the animal data is collected (e.g., name, age, weight, height, activity, and/or associated groups) and the one or more source sensors, which includes at least one characteristic of the one or more source sensors. The at least one characteristic includes at least the sensor type, one or more sensor settings, sensor brand, sensor model, sensor firmware, and the like. In a refinement, the animal data includes metadata that identifies one or more characteristics of the animal data and the one or more source sensors. In some variations, the computing subsystem 22 and/or the wagering system 28 and/or the probability assessment system 30 takes one or more further actions upon the animal data when received. Examples of such further actions include, but are not limited to, steps that normalize, timestamp, aggregate, store, manipulate, denoise, enhance, organize, visualize, analyze, anonymize, synthesize, summarize, replicate, productize, and synchronize the animal data. In a refinement, the one or more actions are transformative to the animal data and/or its one or more derivatives. In another refinement, computing subsystem 22 applies a schema suitable for real-time or near real-time data transfer that reduces latency, provides error checking and a layer of security, and encrypts the animal data or parts thereof. In another refinement, the computing subsystem or the wagering system or the probability assessment system: (1) communicates directly with one or more systems to monitor, receive, and record at least one request for the predictive indicator, the at least one computed asset, and/or the animal data, (2) provides the one or more users (e.g., systems) requesting access to the predictive indicator, the at least one computed asset, and/or the animal data with an ability to make one or more requests for data (e.g., by subject or group of subjects, one or more characteristics, data type, time, and the like); and (3) is operable to send and/or receive data. This may be achieved by utilizing a technology such as blockchain. Utilizing a technology like blockchain, the system may have the ability to monitor animal data, and every transaction associated with the data, starting from when the data is collected by the system. At any given time, a data provider or authorized user within the system can look at the compete historical tree of that individual's data and any given usage of the data which can include where the data was sent, any restrictions attached to the data, and other metadata associated with each data. In another refinement, computing subsystem 22 and/or wagering system 28 and/or probability assessment system 30 are operable to associate at least one request for the predictive indicator, the at least one computed asset, and/or the animal data with at least one user, group of users, or classes of users. For example, the association can be made by the computing subsystem, the wagering system, or the probability assessment system between a system (e.g., its own system) to a person or group of persons or class of users who are making the request for data (e.g., to place a bet), or to another system (e.g., third party system). In another refinement, the computing subsystem, the wagering system, or the probability assessment system can associate the animal data and the one or more requests from a third-party system to a person who is making a bet. Furthermore, the computing subsystem the wagering system or the probability assessment system can associate the at least one request for data with the one or more targeted individuals or groups or targeted individuals the animal data is derived from. For example, if a request is made for animal data from a targeted individual in order to see the targeted individual's real-time blood pressure and heart rate vitals, or the probability that the targeted individual will experience a heart attack in the next n months, the computing subsystem or the wagering system or the probability assessment system can associate the specific request with the animal data, the corresponding targeted individual, and any other data or metadata required to fulfill the request. In another refinement, the animal data is grouped into one or more classifications with each classification having an associated computed asset or value.

In a variation, computing subsystem 22 is operable to manage the one or more source sensors, and one or more data streams from the one or more source sensors, by at least one characteristic from the group consisting of; organization, sensor type, sensor parameter, data type, data quality, timestamp, location, activity, the targeted individual, groupings of targeted individuals, and data reading. In a refinement, the management and/or administration of a sensor can include functionality such as scanning for, and pairing, one or more sensors with the system, assigning one or more sensors (if required) to one or more individuals within the system, assigning the one or more sensors and/or individuals to an organization or event, verifying the one or more source sensors are placed correctly on the subject and streaming desired data once applied on subject, and the like. It can also include functionality to support the real-time or near real-time streaming of the one or more sensors to the system, including an auto-reconnect function when the one or more sensors disconnect or when a lapse in streaming occurs. In addition, the system may provide one or more alerts based on sensor disconnection, sensor failure (including battery failure), sensor degradation (e.g., producing a quality of data that does not meet a minimum established standard or threshold), and the like. In a refinement, the computing subsystem is operable to gather information from the one or more source sensors by communicating directly with the one or more source sensors, its associated cloud, or a native application associated with the one or more source sensors. In another refinement, the computing subsystem is operable to send one or more commands to the one or more sensors to change one or more sensor settings. For example, such commands can cause an individual source sensor to be turned on or off, to a battery savings mode for energy saving, to start or stop streaming, or to increase or decrease the amount of data throughput to accommodate the bandwidth available for streaming. As another example, such commands can increase or decrease the data collection frequency and/or sensor sensitivity gain of the at least one source sensor. In another refinement, computing subsystem 22 is operable to communicate with a plurality of source sensors on a targeted individual or one or more source sensors on multiple targeted individuals simultaneously. In another refinement, computing subsystem 22 synchronizes communication and the one or more data signals or readings from multiple sensors that are in communication with the computing subsystem. This includes the one or more commands sent from the sensor to the system, which may include examples such as a pre-streaming handshake between the sensor and the system to ensure the reliability of both parties, as well as encryption protocols. It also includes synchronization challenges with the one or more data signals or readings. As an example, there may be a mismatch in the timings utilized by each sensor. A sensor's output received by the computing subsystem may be different (for example, by milliseconds) than another sensor even if received by the computing subsystem at the same time. Therefore, the computing subsystem may need to synchronize the data streams to ensure that both streams are aligned.

In another variation, when a request is made from a user for a data type or data set that is not within the computing subsystem 22, the computing subsystem 22 may acquire data from one or more third party organizations, apply or utilize one or more analytics tools (e.g., third party or in-house) on collected or acquired data sets to create requested data to be provided to the one or more users, or create the one or more data types or data sets artificially, which may occur via one or more simulations. Alternatively, computing subsystem 22 may send the animal data to another system (e.g., third-party analytics system for analysis), with the computing subsystem receiving the analyzed data from the third party and providing it to the one or more users. Upon sending the data to another system or source (e.g., a third party wagering system, a third party probability assessment system, a third party analytics company, a wagering system or probability assessment system or analytics system that is part of the computing subsystem), the computing subsystem is operable to record one or more characteristics of the animal data provided as part of its one or more distributions. These characteristics of the animal data may include at least one of sources of the animal data, specific personal attributes of the one or more individuals (e.g., name, weight, height, corresponding identification or reference number), type of sensor used, specific sensor configurations, location, activity, data format, type of data, algorithms used, quality of the data, when the data was collected, associated organization, associated event, and speed at which the data is provided. Alternatively, the receiving party can send animal data that has been sent by the computing subsystem and analyzed by the receiving party directly to the user. In another example, animal data that is inclusive of other data (e.g., non-biological statistics) can be obtained and utilized for analysis. For example, how many points an athlete scores in a game may be obtained from third parties and utilized as part of an analysis that looks at the athlete's heart, respiration rate, and biological fluids to derive an insight or other indicator. In a refinement, the computing subsystem may provide an anonymized data output without any identification or association to an individual or group of individuals. While the animal data is oftentimes associated with an identifiable one or more targeted individuals or groups of targeted individuals, it should be appreciated that one or more inputs or outputs associated with the animal data and its derivatives (which can include computed assets and predictive indicators) can be anonymized or de-identified. De-identification involves the removal of personal identifying information in order to protect personal privacy, but retains characteristics that make the data useful (e.g., in the context of a human, characteristics such as age, weight, height, medical conditions, country of origin, blood type, biological fluid-derived information, and the like). In the context of the present invention, anonymized and de-identified are considered synonymous.

In other variations, the computing subsystem 22 is operable to allow one or more users to choose the frequency with which the predictive indicator, computed asset, animal data and/or its one or more derivatives is provided (e.g., 1 or more packets of data per second, with each packet of data containing specified data). Moreover, computing subsystem 22 can be operable to allow users to select one or more parameters such as latency (e.g., real-time or near real-time vs not) and time period that enable a user to maximize the value of any given data for their specific use case. In some cases, there may not be enough data collected in the computing subsystem for there to be a meaningful predictive indicator to be derived initially; however, the system will be operable to do so. The ability to provide animal data—particularly the predictive indicator—in real-time or near real-time is especially useful for both wagering and probability assessment applications. Such real-time or near real-time data is necessary for computing subsystem 22 and/or wagering system 28 and/or the probability assessment system 30 to provide one or more wagering strategies or markets such as proposition bets (prop bets), creation or modification of a prediction or one or more odds, adjustment or modification of a probability evaluation, a strategy to mitigate or prevent a risk, or other use cases (e.g., real-time health feedback). In another refinement, computing subsystem 22 and/or the wagering system and/or the probability assessment system are operable to allow a user to select at least one characteristic upon which the predictive indicator, computed asset, animal data and/or its one or more derivatives are provided. A characteristic may include the one or more sources of the animal data, specific personal attributes of the one or more individuals or groups of individuals, type of sensor used, sensor properties, classifications, specific sensor configurations, location, activity, data format, type of data, algorithms used, quality of the data, when the data was collected, associated organization, associated event, and speed at which the data is provided.

In some situations, computing subsystem 22 provides and/or uses historical animal data. In a refinement, historical data from one or more similar events for an individual or similar individual(s) may be useful to a user for predicting performance related to any given event. For example, if a gym equipment manufacturer wants to create a predictive indicator aimed at predicting fatigue (or the likelihood that fatigue will occur at any given time based on exercise patterns) for users of their product (e.g., in-home cycling equipment) as part of its platform subscription offering, using historical animal data derived from users in cycling-focused fitness classes may be useful in enabling the manufacturer to create a predictive indicator for any given user in order to predict current or future biological performance while using their equipment. Advantageously, historical data for a given one or more subjects or groups of subjects enables the system to learn from that information and use that learning to provide more precise results (e.g., learning how a subject's heart rate performs for a given activity allows for the system to fine tune its heart rate algorithm to provide a more accurate reading). In another refinement, historical data from one or more similar individuals may be useful to users for predicting performance for any given subject. For example, if an insurance company wants to understand the likelihood of any given subject having a specific medical condition (e.g., heart attack) within a predefined period of time, the insurance company may utilize data from individuals that share one or more characteristics with the individual (e.g., age, height, personal history, social habits, blood type, medical history, prescription history, ECG data history, heart rate history, blood pressure history, genomic/genetic history, biological fluid-derived data history) to run one or more simulations in order to determine a likely outcome for whether or not that subject will experience the medical condition within the requisite period of time. In another refinement, historical data that is not derived from animal data (e.g., in the context of sports, traditional statistics like points, rebounds, assists, goals, shots, win/loss percentage, and the like) may also be used.

Speculation system 10 may be operable to allow a user to adjust one or more parameters within one or more data sets in order to run one or more simulations that can determine (or provide information supporting) whether or not to place a bet, evaluate or calculate a probability for the occurrence of an outcome of an event, make a prediction, modify a previously determined probability for an event, formulate a strategy upon which a market is created for one or more individuals to place a wager on, or recommend an action. In a refinement, speculation system 10 provides one or more artificially-created data sets derived, at least in part, from one or more sensors as an alternative to real data sets. Artificial data can be beneficial in the event a user wants to re-create (e.g., generate) data from previous historical data/events to establish trend lines and aid in the study and understanding of any given performance change for any given subject in light of modifications (e.g., changes) to any given one or more inputs (e.g., variables) in order to predict future occurrences. Upon training the system to understand these one or more performance changes and the variables associated with the changes (e.g., which can occur via one or more neural networks), a user can re-create data (e.g., using one or more methodologies including within one or more simulation scenarios) to predict future events or occurrences based on, for example, a historical performance understanding of the subject, the historical impact of the one or more inputs, current performance, the current impact of the one or more inputs, and trends seen by the system for similar events, subjects, and inputs. Advantageously, the one or more inputs can be biological data. In particular, computing subsystem 22 or the wagering system 28 or the probability assessment system 30 can create one or more artificially-generated animal data sets, computed assets, or predictive indicators, which may occur via one or more simulations that utilize at least a portion of the predictive indicator, computed asset, the real collected animal data, and/or its one or more derivatives. This may occur utilizing one or more artificial intelligence techniques (e.g., one or more trained neural networks, machine learning systems) or statistical models. In a further refinement, a simulation can be comprised of a plurality of simulations (e.g., running n number of simulations simultaneously or in succession to derive a single simulation output). In a variation, the computing subsystem 22 or the wagering system 28 or the probability assessment system 30 can generate simulated data derived from at least a portion of the predictive indicator, the at least one computed asset, and/or the animal data of the one or more targeted individuals or groups of targeted individuals (including one or more anonymized individuals or groups of anonymized individuals). In these contexts, “generate” can be inclusive of “create” and vice versa. “Generate” can also be inclusive of “derive” and vice versa. The computing subsystem or the wagering system or the probability assessment system may be operable to use at least a portion of the simulated data either directly or indirectly: (1) as a market upon which one or more wagers are placed or accepted; (2) to accept one or more wagers; (3) to create, enhance, modify, acquire, offer, or distribute one or more products; (4) to evaluate, calculate, derive, modify, enhance, or communicate one or more predictions, probabilities, or possibilities; (5) to formulate one or more strategies; (6) to take one or more actions; (7) to mitigate or prevent one or more risks; (8) as one or more signals or readings utilized in one or more simulations, computations, or analyses; (9) as part of one or more simulations, an output of which directly or indirectly engages with one or more users; (10) to recommend one or more actions; (11) as one or more core components or supplements to one or more mediums of consumption; (12) in one or more promotions; or (13) a combination thereof.

In addition to sensor data, other types of collected animal or non-animal data can be incorporated into the one or more simulations depending on the use case. For example, personal information may be incorporated and can include age, height, race, country/area of origin, ethnicity, gender, information from medical records, personal history, social history, health history, social habits, education records, criminal history, feelings, psychological evaluations, and the like. For sports, information can include win/loss record and other statistics (e.g., points won/lost, results, individual statistics like points, rebounds, shots, forehands, backhands, and the like). For sports betting, this can also include past wagers, user behaviors, betting trends, or other user data. Characteristically, an ability to add or change one or more inputs (e.g., variables) within a requested data set allows for a user to determine the one or more parameters upon which an artificial data output is generated, while tailoring the output to the one or more specifications of the user. The one or more inputs may include any data relevant for understanding past behaviors to predict future performance including one or more signals or readings from animal data and non-animal data alike. The artificial data output may be, for example, artificial animal data, a computed asset, and/or a predictive indicator. In a variation, the computing subsystem, the wagering system, the probability assessment system, or other system may use or apply at least a portion of the simulated data, either directly or indirectly, to create, enhance, or modify a predictive indicator, at least one computed asset, and/or animal data (which includes any of its derivatives). At least a portion of the created, enhanced, or modified predictive indicator, the at least one computed asset, and/or animal data can then be utilized either directly or indirectly: (1) as a market upon which one or more wagers are placed or accepted; (2) to create, modify, enhance, acquire, offer, or distribute one or more products; (3) to evaluate, calculate, derive, modify, enhance, or communicate one or more predictions, probabilities, or possibilities; (4) to formulate one or more strategies; (5) to take one or more actions; (6) to mitigate or prevent one or more risks; (7) as one or more signals or readings utilized in one or more simulations, computations, or analyses; (8) as part of one or more simulations, an output of which directly or indirectly engages with one or more users; (9) to recommend one or more actions; (10) as one or more core components or supplements to one or more mediums of consumption; (11) in one or more promotions; or (12) a combination thereof. In a refinement, the one or more creations, enhancements, or modifications are dynamic. Advantageously, the one or more simulations, as well as the one or more applications of the output to the one or more use cases, can occur in real-time or near real-time.

In a refinement, one or more inputs/parameters can be changed (e.g., randomized) within a simulation to provide one or more new simulated data sets. In a further refinement, at least a portion of the generated artificial data output can be used as one or more data sets or as part of another one or more data sets utilized within one or more simulations, computations, or analyses. For example, one or more simulations may be nm to determine what an athlete's future biological data (e.g., physiological data, biomechanical data, location data) may look like for any given match. Once the artificial data is generated, the artificial data may be utilized in a further one or more simulations to determine one or more predictions related to whether or not the athlete will win a match. Such determinations can occur in real-time or near real-time. In another refinement, simulated data that incorporates at least one type of animal data may be utilized to fine-tune a predictive indicator. Advantageously, the one or more predictions can be modified or enhanced in real-time or near real-time as new data is collected and further simulations or statistical models or adjusted. For example, the system may conclude based on historical data that an outcome may occur, and the system may also create a prediction related to the outcome occurring. By running one or more simulations using at least a portion of artificial data that incorporates at least one type of animal data, the system can adjust or enhance the predictive indicator to determine a more likely probability of an outcome occurring based upon various data, which could include, for example, current match status of a tennis player (e.g., Player A is in Game 4 of Set 2 and is losing 6-4, 3-2), historical data (e.g., all match results for Player A when he is in Game 4 of Set 2 and is losing 6-4, 3-2), current conditions (e.g., humidity, temperature, elevation), same conditions for when previous matches were played (e.g., humidity, temperature, elevation), related animal data (e.g., exhibited physiological characteristics, biomechanical characteristics, biological fluid-based characteristics, location-based characteristics, and the like) and other types of non-animal data. In another example, an insurance company may have a hypothesis related to the characteristics of one or more individuals and may utilize at least a portion of the animal data to run one or more simulations to determine a likely biological outcome for those individuals. An outcome may be, for example, the likelihood a person will succumb to a disease in the next n months, the likelihood a given injury will achieve a given recovery rate, the likelihood an individual may experience a medical episode (e.g., seizure, heart attack), and the like. Based on the one or more simulations, artificial data may be created, upon which odds for any given outcome may be created or adjusted and provided to a speculation system (e.g., likelihood of an individual having a stroke based on the individual's one or more characteristics). In the insurance example, a premium may be adjusted by the insurance company for individuals with those characteristics. In another example, an elderly care home may utilize at least a portion of the animal data to run one or more simulations to determine a likely health outcome for any given individual, and therefore determine the amount of future care required for that particular individual. Based on the amount of future care likely required, the home may be able to tailor its pricing for each individual based on the individual's profile. In another example, an automotive or aircraft manufacturer may want to run simulations to fine-tune the predictive indicator in order to provide one or more responses related to a subject within the vehicle or aircraft to mitigate or prevent a risk. More specifically, an automotive manufacturer may want to determine whether someone that is exhibiting specific physiological or biomechanical characteristics while driving a vehicle may be at risk for causing an accident. By utilizing the animal data and/or its one or more derivatives, the vehicle may take one or more actions (e.g., stop the car, pull over, drive to the hospital) based upon the predictive indicator and other animal data in order to mitigate or prevent a risk (e.g., the vehicle may drive itself to a hospital if it is determined that the person is having a heart attack based on collected sensor data; the vehicle may stop itself if it is determined that the likelihood of a person having a heart attack with a given profile and characteristics—a specific age, weight range, height range, heart condition, increased heart rate, elevated blood pressure, elevated stress level, irregular biomechanical movements—while holding onto the steering wheel and driving is greater than a predetermined percentage; or a more absolute prediction via the predictive indicator that the subject will have a heart attack with these given sets of characteristics and parameters). In another example, an airline may monitor the real-time biological characteristics of its one or more pilots via one or more source sensors while flying and take one or more actions (e.g., notify the airline, take control away from the pilot, put the plane on autopilot, enable control of the plane to the airline or airline manufacturer remotely) based upon the probability of an occurrence happening related to at least a portion of the animal data.

In another refinement, simulated data that incorporates at least a portion of animal data may be utilized to create one or more prop bets for a simulated event. For example, if a system has previously collected Team A's heart rate vs Team B, the system could create one or more new bets that utilize previously collected data incorporated as part of one or more simulations. The bet could be “Is Team A's Average Max Heart Rate going to exceed 170 beats per minute for the duration of a match vs Team B in 10,000 simulated matches”. In another refinement, simulated data that incorporates at least a portion of animal data may be utilized to create new prop bets for virtual events (including simulated events) or as information utilized as part of a wagering strategy for virtual bets. For example, if the system has collected respiration rate for one or more real subjects (e.g., real horses) in one or more real races, a system could generate simulated data (e.g., simulated respiratory rate) based on the collected respiration rate data from one or more simulated races that would enable the system to create one or more prop bets or betting products for one or more virtual subjects (e.g., virtual horses) that utilizes at least a portion of the generated simulated animal data in one or more virtual races (e.g., the bet could be: “is the virtual horse's max respiration rate in the virtual race going to reach above Indicator X”). In a variation, the simulated data generated may not share the same characteristics from which it was derived from. For example, in the case of generating artificial respiration rate for a virtual horse, the simulation may characterize and display the generated artificial respiration rate as another indicator (e.g., a color, another name such as “fatigue”, and the like). In a further refinement, the one or more virtual subjects share at least one common characteristic to the one or more real subjects, and the virtual event shares at least one common characteristic to the event from which the real animal data was collected (e.g., horse Z ran in a real race, and a virtual horse Z is running in a virtual race, with at least one characteristic of the real horse and the event in the system. This characteristic may be, for example, respiration rate, and the event may be a horse race. Bet: “is the virtual horse Z's max respiration rate in the virtual race going to reach above Indicator X”). Subject characteristics could include biological characteristics, physical characteristics, profile characteristics (e.g., same name, jersey number, team name, team colors), and the like. In another example, a simulation game (e.g., video game) or virtual world video game may create one or more wagers or products (e.g., in-game virtual products for purchase) related to the real animal data of the one or more users playing the game (e.g., utilizing real animal data of the user that is incorporated as part of the virtual video game, creating a reward in the game for the user who reaches a goal while utilizing simulated data that incorporates at least a portion of their animal data within the game; enabling a user to purchase an artificial data-based virtual product that is generated, at least in part, from the animal data; creating a bet type or product based on the artificial animal data utilized in the video game). In yet another refinement, simulated data is created for a virtual event or simulation game (e.g., video game) based upon at least a portion of the animal data, which may create a new value or asset to the wagering and/or probability assessment system. For example, in the scenario above, a user may want to know the probability that Horse Z wins the race in a simulated event when its simulated respiration rate goes above Indicator X, and how often this occurs in any given simulated race. The system may utilize various data including at least a portion of animal data to generate the simulated data (e.g., respiration rate of Horse Z collected from one or more source sensors for every available race; respiration rate of other horses from one or more source sensors if available; simulated respiratory rate data generated from one or more simulated races; other factors collected in the real world that may be utilized as inputs for the simulated races—environmental conditions like weather or temperature, injuries, biological fluid data, and the like). Based on the information, a user may place a bet on the virtual horse race. In still another refinement, simulated data that incorporates at least one type of animal data may be utilized to create or adjust betting lines (e.g., adjust odds) with more precision. For example, if a line is set for Player A vs Player B for a particular match, the computing subsystem may run one or more simulations using similar match conditions (e.g., on-court temperature, previous win-loss record) and an input that incorporates at least one animal data input of the Player (e.g., all Player A animal data vs Player B animal data), enabling the system to determine the probability of an outcome with greater precision. The probability may then be utilized to create or adjust one or more odds, which can occur in real-time or near real-time.

In a refinement, one or more artificial data sets can be generated, either randomly or otherwise, subject to one or more parameters set by the user. This may be useful, for example, in the event that the real data a user desires cannot be acquired, captured, or created. In the case where a user has a request that may not make it feasible to acquire real data (e.g., the requested data cannot be acquired in a requested timeframe, the consideration associated to the real data sets is too costly, the use case required by the acquirer necessitates one or more data sets that are not found within the system or not obtainable), speculation system 10 may generate artificial data that conforms to the one or more parameters established by the user, which may be made available for product creation, adjustment, enhancement, acquisition, distribution, and/or consumption. The new one or more artificial data sets may be created by application of one or more artificial intelligence techniques that can analyze one or more previously captured data sets that match at least one of the characteristics required by the acquirer. The one or more artificial intelligence techniques (e.g., machine learning-based engine, one or more neural networks) will recognize patterns in the one or more real data sets and create artificial data that matches or meets the minimum request of the user (e.g., the wagering entity, bettor, organization evaluating a probability or creating a strategy to mitigate or prevent a risk, platform providing a recommendation or information to take an action). The one or more data sets can be created based on a single individual, a group of one or more individuals with one or more similar characteristics, a random selection of one or more individuals within a defined group of one or more characteristics, a random selection of one or more characteristics within a defined group of one or more individuals, a defined selection of one or more individuals within a defined group of one or more characteristics, or a defined selection of one or more characteristics within a defined group of one or more individuals. In a refinement, a group can include a plurality of groups. Based on the user's requirements, the speculation system may have the ability to isolate a single variable or multiple variables for repeatability in creating one or more artificial data sets in order to keep the data both relevant and random.

Another method for creating an artificial data set involves extending a data set of a previously collected real data set with simulated data. For example, a system that has access to a specified quantity of in-play/match data for Athlete A, (e.g., 10, 100, 1000, or more hours) which includes different types of data and metadata (e.g., in the context of a sport like tennis, on-court temperature, humidity, heart rate, miles run, swing speed, energy level, respiration rate, muscle activity, hydration levels, biological fluid-derived data, shot power, length of points, court positioning, opponent, opponent's performance in specific environmental conditions, winning percentage against opponent, winning % against opponent in similar environmental conditions, current match statistics, historical match statistics based on performance trends in the match, date, timestamps, points won/lost, score), can extend the data set using one or more artificial intelligence techniques by recreating at least a portion of an event (e.g., a match) in which the given athlete may not have even played and/or generate artificial data for Athlete A within the recreated event (e.g., Athlete A played a 2-hour tennis match with heart rate data captured but a user wants heart rate data for the 3^{rd} hour of a match that was never played and will be played in the future. Therefore, the computing subsystem can run one or more simulations to create the data). More specifically, one or more neural networks may be trained with one or more of these data sets to understand biological functions of Athlete A and how one or more variables can affect any given biological function. The neural network can be further trained to understand what outcome (or outcomes) occurred based on the one or more biological functions and the impact of the one or more variables, enabling correlative and causative analysis. For example, upon being trained to understand information such as the one or more biological functions of Athlete A within any given scenario including the present scenario, the one or more variables that may impact the one or more biological functions of Athlete A within any given scenario including the present scenario, the one or more outcomes that have previously occurred in any given scenario including the present scenario based on the one or more biological functions exhibited by of Athlete A and/or the one or more variables present, the one or more biological functions of athletes similar and dissimilar to Athlete A in any given scenario including scenarios similar to the present scenario, the one or more other variables that may impact the one or more biological functions of Athlete A in any given scenario including scenarios similar to the present scenario, the one or more variables that may impact the one or more biological functions of other athletes similar and dissimilar to Athlete A in any given scenario including scenarios similar to the present scenario, and the one or more outcomes that have previously occurred in any given scenario including scenarios similar to the present scenario based on the one or more biological functions exhibited by athletes similar and dissimilar to Athlete A and/or the one or more variables, an acquirer of data may request one or more simulations to be run to extend the current collected data set with artificially-generated data (e.g., Athlete A just played 2 hours with various biological data including location-based data captured. An acquirer wants location-based data for the 3^{rd} hour under the same match conditions, so the system may run one or more simulations to create the data based on previously collected data) or predict an outcome occurring for any given activity (e.g., the likelihood of Athlete A winning the match in the last set vs Athlete B, based on looking only at Athlete A's data) via the predictive indicator. In a variation, the one or more neural networks may be trained with multiple animals (e.g., athletes), which may be on a team, in a group, or in competition with one another, and the one or more neural networks may be trained with one or more data sets from each animal to more accurately generate a predictive indicator to predict one or more outcomes (e.g., whether Athlete A will win the match vs Athlete B). In this example, the one or more simulations may be run to first generate artificial sensor data based on real sensor data for each athlete, and then utilize at least a portion of the generated artificial sensor data in one or more further simulations to determine the likelihood of any given outcome and/or make a prediction.

In another example, a sports team may want to determine the right time to take an athlete off the court during a game, or a hospital may want to determine whether it should continue to allow a surgeon to operate after working a certain number of hours in a day or week. By running one or more simulations utilizing at least a portion of the real animal data, the sports team or hospital will be able generate one or more artificial data sets from which one or more predictive indicators can be derived that will enable a determination by the sports team or hospital as to whether or not to take an action (e.g., in the case of the sports team, the consideration is whether to allow the athlete to stay on the court while he is still performing at a high level, knowing that fatigue could lead to injury or a decline in performance at a sub-optimal future time, or to take him out of a game; in the case of the hospital, the wager is whether to allow the surgeon to continue to operate after working for a certain amount of time continuously or while exhibiting specific characteristics, with one risk being human life and one benefit being experience which can lead to saving more lives). In yet another example, a remote patient monitoring or telehealth platform may want to provide both the medical professional (e.g., doctor) and patient with the likelihood of a patient experiencing any future medical condition (e.g., flu, heart attack, diabetes, stroke) based upon their one or more real-time vitals provided to the application via one or more source sensors (e.g., heart rate, ECG, blood pressure, fatigue, stress, sleep data) as well as other data (e.g., nutrition, age, weight, height, medical history, biological fluid-based data history, genetic/genomic history, prescription history). By running one or more simulations utilizing at least a portion of the real animal data, the medical professional or other administrator (e.g., the speculation system) can generate one or more artificial data sets from which one or more predictive indicators can be derived. The predictive indicator may provide, for example, the medical professional and patient with the likelihood of an occurrence happening (e.g., % chance the patient may have a stroke in the next 6 months based on analysis of their animal data and other data; based on one or more characteristics of the patient, the patient will experience p medical condition in the next 30 days unless the x, y, z steps are taken), as well as a recommended action to mitigate a risk (e.g., reduce stress by walking in minutes a day; keep eating specific foods in order to keep blood pressures low). In a refinement, the speculation system may be programmed to provide one or more alerts based on one or more readings related to the predictive indicator, computed asset, animal data, and/or its one or more derivatives. For example, alerts based on a subject achieving a maximum heart rate or reaching a pre-defined “energy level” that warrants an alert, or the system detecting an irregularity in ECG data. In this example, detection of such anomalies from a subject can occur utilizing historical ECG information gathered from the subject by the system, as well as one or more subjects that share one or more characteristics with the subject (e.g., age, weight, height, medical conditions, and the like). The system can be operable to use historical data as a baseline to detect anomalies in any given subject and provide insights related to any changes in morphology or any other relevant findings. In another refinement, the medical professional or system may take one or more actions based upon the predictive indicator. For example, if the system provides a generated predictive indicator that says a specific patient with diabetes and a wearable sensor (e.g., insulin pump) has a relatively high likelihood of experiencing a significant physical reaction (e.g., going into a coma) based on biological fluid readings (e.g., low blood-sugar levels) with that data communicated to the system, at least in part, by a sensor, or will experience a significant physical reaction in the next n hours if insulin is not administered, the system may be operable to communicate with the insulin pump directly and provide one or more commands to administer insulin to the body via the pump, or generate a nutrition plan based upon the predictive indicator in order to keep blood sugar regulated. Additional details related to a biological data tracking system utilizing a web browser-based application are disclosed in U.S. patent Ser. No. 16/274,701 filed Feb. 13, 2019 and U.S. Pat. No. PCT/US20/18063 filed Feb. 13, 2020, the entire disclosures of which is hereby incorporated by reference.

In another method for creating a simulated data set, previously captured data is re-run through one or more simulations to create the one or more new data sets. For example, if a computing system utilizes a statistical model or a neural network like Long Short-Term Memory (LSTM), and a user wants to create artificial heart rate data for Athlete A that utilizes at least a portion of Athlete A's heart rate data (e.g., characteristics of Athlete A's heart rate data or beats per minute for a variety of scenarios) to incorporate in a simulation (e.g., simulation game such as a video game), the speculation subsystem could be trained utilizing Athlete A's real heart rate data to generate an artificial data set based in part on at least a portion of Athlete A's real heart rate data which could be incorporated into the simulation game. The simulation game could feature, for example, one or more biological metrics (e.g., heart rate, foot speed, swing speed) for Athlete A derived from at least a portion of Athlete A's real animal data and/or its one or more derivatives. The simulated data (e.g., simulated biological metrics) for the virtual subject may be based on historical data collected from the real-world subject, or may be displayed in real-time or near real-time for the virtual subject based on real-time or near-real time data being provided to the speculation system by the real world subject and converted into a new artificial data set by the speculation system. In a variation, the indices established by the one or more simulations may be different from the real-world data but converted to be applicable to the simulation (e.g., heart rate data may be converted into color within the game, or a virtual currency may be tied to maintaining or exceeding a heart rate within a specific zone). Advantageously, a predictive indicator can be made available to a user within the simulation game (e.g., an “energy level” bar which can indicate when a virtual subject is tired and predicts when a subject within the game will run out of energy, or when an action by the user needs to be taken based on the likelihood that an occurrence will happen). In a refinement, the predictive indicator is made available for purchase (or tied to consideration) within the game. In some cases, the predictive indicator may need to be adjusted or modified in order to conform to the one or more parameters of the simulation and integrate the data into the simulation. Utilizing this method, probabilities of various outcomes can also be examined. For example, Athlete A's existing data (e.g., previously collected or captured) and the system's ability to run one or more simulations to create one or more new data sets that can be used to determine a probability of a particular outcome. In a refinement, one or more simulations can be run incorporating future-looking data (e.g., artificially-generated data) within another one or more simulations to predict an outcome or create a probability. For example, a person's future heart rate data or biological fluid-based data in a given scenario may be generated based on collected data, with that artificial heart rate data or biological fluid-based data being utilized in a further one or more simulations in order to predict another future outcome (e.g., based on that future heart rate data and/or the one or more biological fluid-based readings, will an outcome occur vs not).

In a variation for creating one or more simulated data sets, existing data with one or more randomized variables is re-run through one or more simulations to create new data sets not previously seen by the system. Utilizing this method as well as previously described examples of inputs, one or more predictive indicators can be generated. For example, when the speculation system has data sets for a specific individual (e.g., athlete) and a specific event (e.g., match the athlete has played), the speculation system may have the ability to re-create and/or change one or more variables within the data set (e.g., the elevation, on-court temperature, humidity) and re-run the one or more events via one or more simulations to generate a targeted simulated data output. For example, in the context of tennis, an acquirer may want 1 hour of Player A's heart rate data when the temperature is at or above 95 degrees for the entirety of a two-hour match. The system may have one or more sets of heart rate data at different temperatures (e.g., 85, 91, 94) as well as previously described inputs for Player A in similar conditions as well as other similar and dissimilar athletes in similar and dissimilar conditions. Heart rate data for Player A at or above 95 degrees has never been collected so the system can run one or more simulations to create it, and then utilize that data in one or more further simulations. In another example, the acquirer may want a predictive indicator providing the likelihood that Player A will win the match. In a refinement, the system may also be programmable to combine dissimilar data sets to create or re-create one or more new data sets. For example, a user may want 1 hour of Player A's heart rate data when the temperature is above 95 degrees for the entirety of a two-hour match for a specific tournament, where one or more features such as elevation may impact performance. While this data has never been collected in its entirety, different data sets can comprise the requested data (e.g., one or more data sets from Player A featuring heart rate, one or more data sets from Player A featuring playing tennis in temperatures above 95 degrees, one or more data sets at the required tournament with requested features such as elevation). The system may identify these requested parameters within the data sets and across data sets and run one or more simulations to create one or more new artificial data sets that fulfill the user's request (which may be, for example, a predictive indicator, computed asset, or artificial animal data) based on these dissimilar sets of data. In a variation, the dissimilar sets of data that are used to create or re-create one or more new data sets may feature one or more different subjects that share at least one common characteristic with the target subject (which can include, for example, age range, weight range, height range, sex, similar or dissimilar biological characteristics, and the like). Using the example above, while heart rate data may be utilized for Player A, the system may utilize another one or more data sets from Players b, c, d, which have been selected based upon its relevancy to the desired data set (e.g., some or all of the players may have demonstrated similar heart rate patterns to Player A; some or all of the players have similar biological fluid-derived readings to Player A; some or all of the players may have data sets collected by the system that feature tennis being played in temperatures above 95 degrees). These one or more data sets may act as inputs within the one or more simulations to more accurately predict Player A's heart rate under the desired conditions.

In still another method for simulated data, artificial data sets that are generic in nature (e.g., lacking shared biological parameters) are created. In a variation, one or more randomized data sets are created, with the one or more variables selected by the system rather than the acquirer. This may be particularly useful if, for example, an insurance company is looking for a specific data set (e.g., 1,000,000 smokers) amongst a random sample (e.g., no defined age or medical history, which may be selected at random by the system), or if a wagering company is looking to create one or more new markets (e.g., prop bets) for events that never existed (e.g., prop bet around a video game simulation outcome). In a refinement, one or more artificial data sets are created based on a predetermined number of individuals picked by a given user of the system. In another refinement, one or more artificial data sets are created from a predetermined number of individuals picked at random by the speculation system.

Additional details related to systems for generating simulated animal data and models are disclosed in U.S. Pat. No. 62/897,064 filed Sep. 6, 2019; the entire disclosure of which is hereby incorporated by reference. The present invention is not limited to the type of one or more statistical models or artificial intelligence techniques utilized (e.g., machine learning models, deep learning techniques). Given that the present invention is not limited by any particular application for using simulated data, such data can be used as a baseline or input to test, change, and/or modify one or more sensors, algorithms, outputs, and/or hypotheses. Moreover, data generated from one or more simulations can be used for a wide array of use cases including as a control set for identifying issues/patterns in real data, as an input in further simulations, or as an input to artificial intelligence or machine learning models as test sets, training sets, or sets with identifiable patterns. This artificial data can be used to run simulation scenarios, the use cases of which can range from training to improving performance and the like. For example, an artificial data set created based on real animal data from a particular athlete can be modified using the speculation system to introduce one or more deviations in the data corresponding to characteristics like fatigue or rapid heart rate changes. With this modified data, one or more simulations can be run to see how an individual (e.g., the athlete, the solider, the patient) will perform in, as an example, high-stress situations or in certain environmental conditions (e.g., high altitude, high on-court temperature). This could be particularly useful in fitness applications, insurance applications, and the like. In the case of a human (e.g., athlete) or other animal, with the system establishing the patterns between biological metrics (e.g., heart rate, respiration, location data, biomechanical data) and the likelihood of an occurrence happening (e.g., winning a particular match, maintaining biological functions at a certain or specified level), the speculation system can calculate one or more probabilities of certain conditional scenarios (e.g., “what-it” scenarios and likely outcomes). As an example, the system creating the artificial data can be operable to run multiple simulations in real-time or near real time for any given event (e.g., tennis match) that may be occurring live at any given time, using n number of data inputs in the one or more simulations. Based on the results of those simulations, the system can assign a probability to a given outcome occurring. For example, if the desired analysis is “Will Player A's HR reach 200 in current match,” the system can create a probability of this outcome happening by running one or more simulations, which may include any number of scenarios (e.g., Player B wins the first set and Player A starts feeling stress, fatigue, and muscle tightness in specific areas of the body; air temperature and humidity increase during the match by n degrees and impacts Player A). There can be n number of such simulation scenarios and additionally simulation scenarios may be created on the fly (i.e., dynamically) via the speculation system's ML/AI engine based on, for example, past similar matches. Once the simulations are run the output is collected and analyzed, the system may be set up to provide one or more probabilities related to the outcome under study. In a variation, more than one simulation may occur during the course of any event, with a different output (e.g., probability) resulting based on changes to the one or more inputs or factors (e.g., time). For example, a system that runs one or more simulations to provide Player A with a n % chance of winning a match may run one or more future simulations at a future time (e.g., 10 seconds after the first simulation, 5 minutes after the first simulation, 1 hour after the first simulation, etc.) that may provide a different probability (e.g., a simulation that is run 30 seconds after the first simulation and utilize “score” and “stress” as a portion of the one or more inputs may result in a revised 52% chance for Player A to win the match because Player A lost a game within that time period and has a higher than normal stress level, which has been shown to cause a decrease in performance in previous matches).

In another embodiment, the speculation system may be utilized as a tool to test, establish, and/or verify the accuracy, consistency and/or reliability of a sensor or connected device. Sensors that produce similarly labeled outputs (e.g., heart rate) may use different components (e.g., hardware, algorithms) to derive their output (e.g., heart rate sensors from different manufacturers, or different heart rate sensors from the same manufacturer that utilize different data collection methods or algorithms to produce the “same” output). This means that, for example, an output like heart rate from one device may not be the same as heart rate from another device. The speculation system's ability to bypass a native application or aggregate the data and act upon the data, including normalizing and/or syncing the data, ensures a user has the ability, if desired, to do a relative “apples-to-apples” comparison and compare each sensor output and their corresponding hardware/firmware and algorithm(s) that derive each output (e.g., raw data, processed data), while providing context for the data (e.g., the activity upon which the data was collected) and eliminating other variables (e.g., transmission-related, software-related) that may impact the output. Testing and comparing each sensor or connected device hardware, algorithm(s), or output impartially (e.g., against a designated standard) ensures quantifiable results that have been isolated to the particular component being evaluated. An ability to obtain quantified results for each sensor type and its corresponding components enables a user to select a particular sensor and/or algorithm for all participants of a given group based upon any given requirements or use cases (e.g., wagering, probability evaluation or calculation, product creation or acquisition, or risk mitigation use cases). For example, one sensor manufacturer may provide a more suitable sensor to use for a specific use case compared to another sensor manufacturer. An ability to select components (e.g., sensor type corresponding algorithms) removes key sensor-related variables typically found when using different or inferior components (e.g., different sensors capturing the “same” output or different algorithms). This methodology also ensures an ability to isolate one or more other variables (e.g., differences between the one or more individual subjects). This methodology ensures a trust in the data by a user, and provides organizations creating markets or wagering strategies, probability evaluations, products, recommendations, or risk mitigation or prevention strategies with a quantifiable way to select the proper sensor(s) for their requirements.

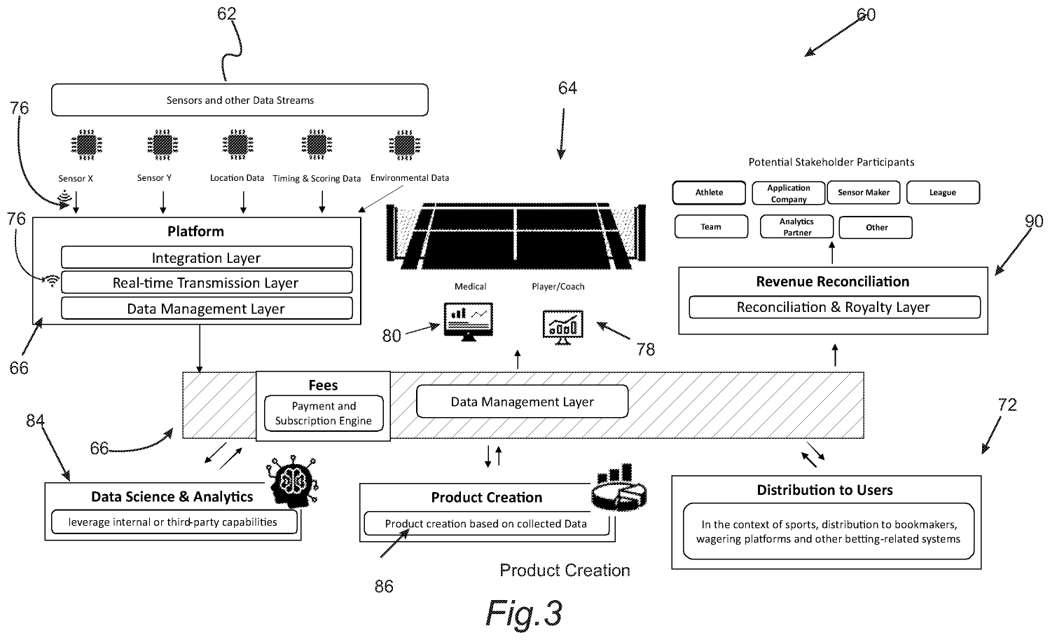

FIG. 3 is a high level, basic overview of the speculation system applied to an event involving sports wagering. Sports speculation system 60 is an example of the systems set forth in the descriptions above in relation to FIGS. 1 and 2. Sports speculation system 60 includes one or more source sensors 62 that collect animal data from one or more targeted individuals engaged in a sports activity in sporting venue 64. Also, while a tennis venue is depicted in FIG. 3, the present design is applicable to any venue, including any sports venue, and applicable to any number of industries. Examples of such sporting venues include, but are not limited to, baseball venues, football venues, basketball venues, soccer venues, ice hockey venues, track & field venues, open race venues or courses including cycling, triathlons, or auto racing, volleyball venues, horse racing venues, dog racing venues, and the like. In a refinement, venue 64 can be non-sports venues, including gyms, homes, fitness studios, manufacturing plants, hospitals, construction sites, and the like. As set forth above, the animal data can be transmitted electronically via wireless and wired connections. Transmission subsystem 76 provides transmission of the animal data to the computing subsystem 66. As noted previously, transmission subsystem 76 and/or its one or more components may be part of computing subsystem 66, may be external to the computing subsystem, or may be integral to one or more of the source sensors. Computing subsystem 66 receives the animal data. As set forth above, at least a portion of the animal data is transformed by the computing subsystem 66 and/or the one or more source sensors 62 into at least one computed asset assigned to a selected targeted individual or a group of targeted individuals. Computing subsystem 66 is operable to transform the at least one computed asset into a predictive indicator. Transformation of the animal data into either a computing asset or predictive indicator can occur via analytics feature 84. Analytics feature 84 can be one or more analytics subsystems, tools, and the like that are part of computing subsystem 66, separate from the computing subsystem and operated by the entity operating the speculation system, separate from the computing subsystem and operated by a different entity (e.g., third party) than the entity operating the speculation system, or operated by an entity (e.g., third party) that analyzes the data and provides at least a portion of the data and/or its one or more derivatives back to the computing subsystem. Analytics feature 84 may utilize one or more statistical models and/or artificial intelligence techniques to transform such data. In a refinement, analytics feature 84 may be operable to create, modify, or enhance one or more products from at least a portion of the Output Information and provide the one or more products to one or more users. In another refinement, analytics feature 84 may be operable to provide at least a portion of the Output Information to one or more users.

Still referring to FIG. 3, the computing subsystem 66 is operable to provide (e.g., offer, distribute, make available, and the like) the predictive indicator, the at least one computed asset, at least a portion of the animal data, its one or more derivatives, and/or a combination thereof (collectively referred to as the “Output Information”) to one or more users through data distribution feature 72. Computing subsystem 66 is operable to provide data to one or more persons, individuals or systems (e.g., platforms, applications), including those directly involved in the event. In the context of sports, this can include coaches and medical personnel 78, as well as analysts, administrators, players, trainers, nutritionists, and other relevant personnel. Advantageously, the Output Information can be provided in real-time or near real-time. In a refinement, the Output Information from computing subsystem 66 can be used to develop wagering or probability assessment products, or provided to other entities to develop wagering or probability assessment products as depicted via product feature 86. Products can include probability-based products, risk mitigation products, animal monitoring applications (e.g., human performance monitoring applications), recommendation products, wagering stimulation products, bet information products, new bet types, and the like. Product feature 86 can be one or more product subsystems, tools, and the like that are part of computing subsystem 66, separate from the computing subsystem and operated by the entity operating the speculation system, separate from the computing subsystem and operated by a different entity (e.g., third party) than the entity operating the speculation system, or operated by an entity (e.g., third party) that creates the one or more products or systems (e.g., applications) and provides the one or more products back to the computing subsystem for distribution. In another refinement, the one or more product subsystems may be operable to provide one or more products and/or at least a portion of the Output Information to one or more users. Finally, FIG. 3 illustrates revenue reconciliation feature 90 in which consideration can be distributed to one or more stakeholders for their contribution in creating, collecting, modifying, enhancing, analyzing, offering, distributing, and/or productizing the animal data or operating the speculation system or any components thereof. In a refinement, transmission subsystem 76, analytics feature 84, product feature 86, distribution feature 72, distribution end points 78, and revenue reconciliation feature 90, or any combination thereof, can be part of computing subsystem 66.

As depicted in FIG. 3, computing system 66 executes the speculation program. When implemented, the speculation program is defined by an integration layer, a transmission layer, and a data management layer. With respect to the integration layer, a user or administrator of the one or more sensors enables the system to gather information from the one or more sensors in one of two ways: (1) the system communicates directly with a sensor, thereby bypassing any native system that is associated with the sensor; or (2) the system communicates with the cloud or native system associated with the sensor, or other system that is storing the sensor data, via an API or other mechanism to collect the data into the system's database. Direct sensor communication is achieved by either creation of new code to communicate with the sensor or the sensor manufacturer writes code to function with the system. The system may create a standard for communication to the system that multiple sensor manufacturers may follow. Communication between the system and the sensor may be a two-way communication where the system can receive data and send one or more commands to the sensor. For example, the system may send one or more commands to the one or more sensors to change one or more functionalities of a sensor (e.g., change the gain, power mode, or sampling rate, start/stop streaming, update the firmware). In some cases, a sensor may have multiple sensors within a device (e.g., accelerometer, gyroscope, ECG, etc.) which may be controlled by the system. This includes one or more sensors being turned on or off, and increasing or decreasing sampling frequency or sensitivity gain. Advantageously, the system's ability to communicate directly with the one or more sensors also enables real-time or near real-time collection of the sensor data from the sensor to the system. The system may have the ability to control any number of sensors, any number of functionalities, and stream any number of sensors on any number of targeted individuals through the single system.

With respect to the transmission layer, a byproduct of the system's direct communication with the sensor is that the system is operable to elongate the transmission signal of the sensor for real-time or near real-time communication, thereby increasing the communication distance between sensor and system, amplifying the receiving connection, and extending the range limitation of the one or more sensors one or more transmission protocols. This can be achieved by utilizing a transmission system that enables the system to communicate with, and utilize, any low power or standard transmission hardware found within the sensor itself (e.g., Bluetooth, BLE, Zigbee, WIFI, cellular communication, Ant+, and the like). Another byproduct of the system's direct communication with the sensor is that a single transmission system can synchronize the communication of real-time or near real-time streaming for multiple sensors that are communicating with the system directly, and act upon the data itself, either sending it somewhere or storing it for later use. This can occur for a single individual or a plurality of individuals. The transmission system can be configured any number of ways, take on various form factors, be located in any number of locations, use one or more transmission/communication protocols or networks (e.g., Bluetooth, ZigBee, WIFI, cellular networks, and the like), be utilized in a variety of environments, and have functionality in addition to simply transmitting data from the sensor to the system (e.g., summarizing, synthesizing or analyzing the data based on use case requirements). Advantageously, the system's direct communication with the sensors via the transmission system also enables real-time or near real-time streaming, particularly in hostile environments where potential interference or radio frequencies from other communications may be an issue.

With respect to the data management layer, the data management layer manages all data (including its one or more derivatives), its properties, its associations (e.g., who/what the data is associated with), and data-related functions (e.g., normalization, synchronization, distribution, etc.). The sensor data that enters the system is in one of the following structures: raw (no manipulation of the data) or processed (manipulated). The system may house one or more algorithms or other logic that deploy data noise filtering, data recovery techniques, and/or extraction or prediction techniques to extract the relevant “good” sensor data from all the sensor data (both “good” and “bad”) collected, or create artificial “good” values in the event at least a portion of the sensor data is “bad.” The system may be programmed to communicate with one or more sensors simultaneously on either a single subject or a plurality of subjects, as well as have the ability to dc-duplicate them in order to transmit enough information for receiving parties to re-structure where the data is coming from and who is wearing what sensor. For clarification purposes, this means providing the system receiving the data with metadata to identify characteristics of the data—for example, a given data set belongs to timestamp A, sensor B, and subject C. In addition, the system can have functionality to associate one or more sensors to one or more users. Once received by the computing subsystem, the sensor data will be sent to either the system cloud or stay local on the system's server depending on the request made. The sensor data that enters the system is synchronized and tagged by the system with information (e.g., metadata) related to the user or characteristics of the sensor including timestamps, sensor type, and sensor settings, along with one or more other characteristics within the system. For example, the sensor data may be assigned to a specific user. The sensor data may also be assigned to a specific event that the user is participating in (e.g., a person playing basketball in Game X of League Y in Season Z), or a general class of activities that an acquirer of data would be interested in obtaining (e.g., group cycling data). The system may synchronize the one or more time stamps with other data sources (e.g., timestamps related to the official time game clock in a basketball game, timestamps related to points scored, etc.). The system, which may be schema-less and designed to ingest any type of data, will categorize the data by one or more characteristics including data type (e.g., ECG, EMG) and data structure. The system may take one or more further transformative actions upon the sensor data once it enters the system including normalize, timestamp, aggregate, store, manipulate, denoise, enhance, organize, analyze, anonymize, synthesize, replicate, summarize, productize, and/or synchronize. This will ensure consistency across disparate data sets. These processes may occur in real-time, near real-time, or on a non-real time basis depending on the use case and requirements of the user. Given the large influx of data streaming or provided from the one or more sensors, which may be significant in volume, the system may also utilize a data management process that may include a hybrid approach of unstructured data and structured data schemas and formats. Additionally, the synchronization of all incoming data may use specific schema suitable for real-time or near real-time data transfer, reducing latency, providing error checking and a layer of security with an ability to encrypt parts of a data packet or the entire data packet. The system will communicate directly with other systems to monitor, receive, and record all requests for sensor data, and provide organizations that seek access to the sensor data with an ability to make one or more specific requests for data that is required for their use case. For example, one request may be for 10 minutes of real-time heart rate for a specific individual at a rate of 1× per second. The system will also be able to associate those requests with one or more users or one or more groups/classes of users.

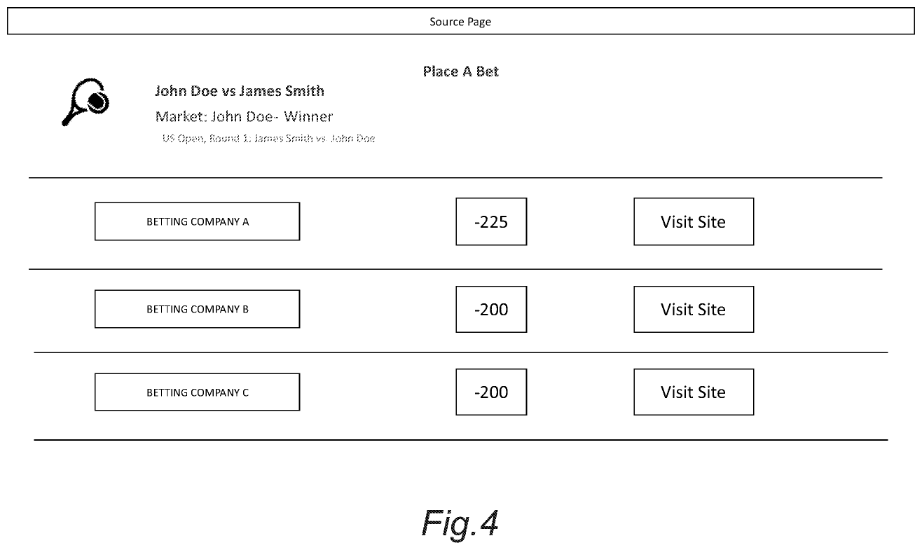

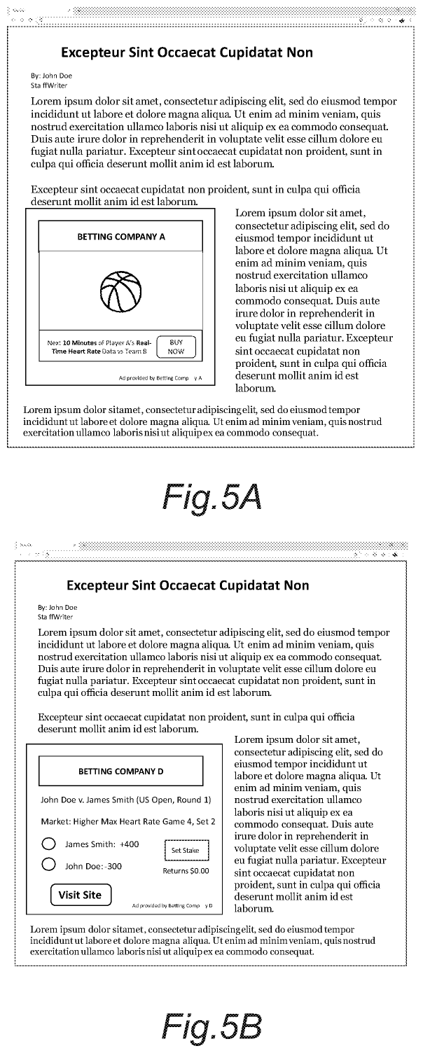

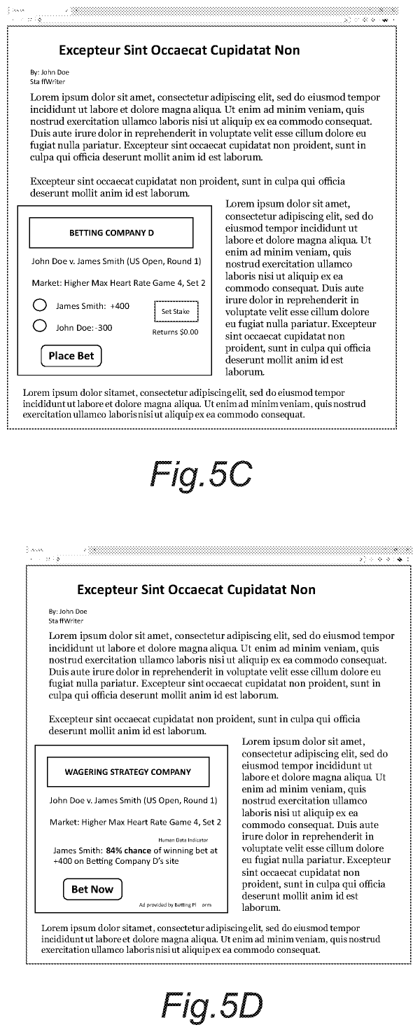

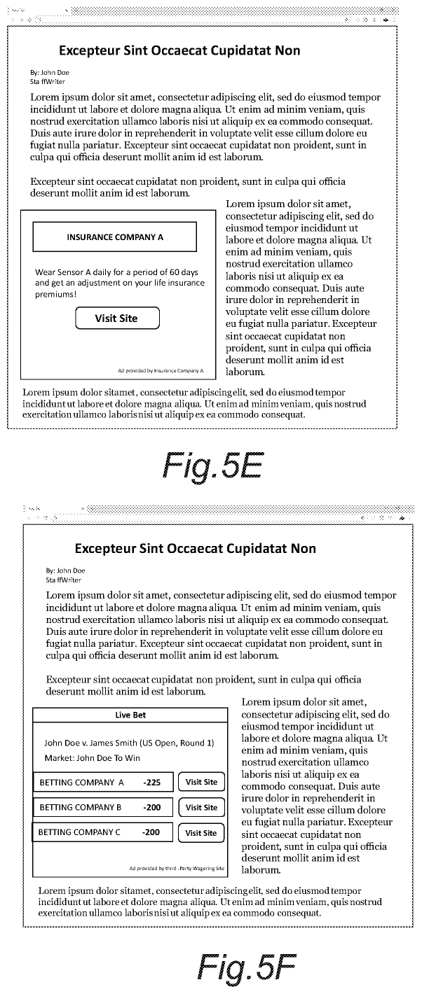

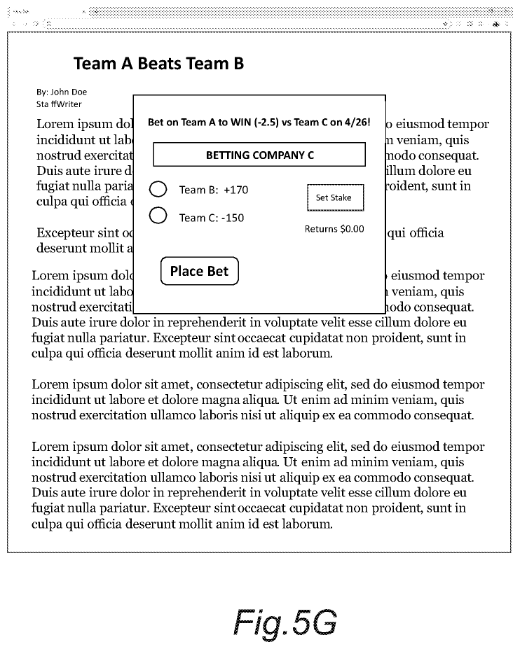

Another aspect of an effective speculation system is usage of the animal data in commerce, which includes one or more promotions (e.g., advertisements, engagements) related to one or more animal data-derived products or services created and/or provided (e.g., offered, distributed, made available, and the like) by the system or one or more third parties that engage one or more users. For example, animal data may be utilized, either directly or indirectly, within a promotion on a web page or other digital platform for the purpose of attracting a user to click through to a web page (e.g., a third party web page) or other digital destination that directly or indirectly utilizes the animal data. For web services, one way to accomplish this is by utilizing an inline frame (Iframe), which can be an HTML document embedded inside another HTML document on a website. An Iframe can be used to insert content from another source, such as an advertisement or engagement (e.g., wagering opportunity, informative literature), into the web page. In some cases, the Iframe or widget is utilized to increase a user's time spent on a web page or other digital destination that feature display ads that refresh for a specified period of time (e.g., every 15 seconds), as well as to target a user to click through to another destination, which in some cases is a third-party site, to provide (e.g., sell) the user with a service, product, or benefit in exchange for consideration. In addition, increased time spent on a page typically leads to more highly engaged users which can lead to repeat visits to a site and more click throughs. There are other methods to serve in the third-party widget (e.g., JavaScript), and the present invention is not limited by these other methodologies used. FIG. 4 provides an example of an advertisement that can be displayed in an Iframe. FIGS. 5A-5G, in addition to FIG. 4, provide examples of particular types of promotions for animal data acquired by the speculation systems set forth above. Note that the invention is not limited to the type of display device utilized to display the promotion and may include one or more monitors, mobile device, a smartwatch, or within smart glasses or eyewear where the promotion can be visualized. Note that while these particular examples of promotions are rendered in a web page to communicate in visual form, promotions can be communicated in other ways including via an audio or aural format (e.g., verbal communication of an advertisement). In another embodiment, other digital platforms that utilize at least a portion of the animal data for advertisement or user engagement purposes (e.g., wagering) include virtual reality systems and augmented reality systems.

For a user (e.g., fan) engagement system such as an augmented reality system or virtual reality system, the speculation system can provide a person engaging with media (e.g., watching a live event such as a sporting event) with an ability to see and interact with the animal data. This may be part of, for example, the in-venue experience or at-home experience. The animal data can either be utilized within the fan engagement system to place one or more bets (e.g., a user can view an athlete's energy level within the fan engagement system as the athlete is playing in a match and a user places a bet on the match through the fan engagement system at any given time, such as during the match when the player looks tired), utilized within the fan engagement system to enable one or more bets to be placed outside of the fan engagement system (e.g., a user sees the player's energy level through the augmented reality system and a user places a bet on the match on the user's mobile device), or provides information to stimulate a person to place a bet (e.g., a user sees the predictive indicator for a player through the augmented reality system and decides to place a bet). The animal data may also have other visuals within the fan engagement system associated with it (e.g., brands in a sponsorship) to further exploit the value of the animal data within the fan engagement system.

For the speculation system to provide animal data to a fan engagement system such as an augmented reality system for wagering, the system may first use object recognition and tracking around a specified area (e.g., within the context of sports, around a field of play area including stadiums and fields with known boundaries and fixed objects). The system may then create an inventory of known identified scenes and tracking information along with the ability to update this information as and when required. The system may acquire known imagery data sets available to help fill in the gaps in this inventory. Using sports as an example (but not limited to sports), the AR system may use 3D tracking for the players and ancillary objects (e.g., tracking ball movement). Based on the position of the player with respect to playing field and other players, augmented objects may be placed such that the visualization is relevant to the play. Additional data from sensors like location-based data (GPS), directional sensors, accelerometers, etc. may be used to fine-tune the placement of players and bring other data points like elevation and latitude into the calculation of 3D models. The system may also look for features in the environment around the fixed known objects, and by tracking the changes in those objects with respect to some fixed point, will try to recognize and substitute relevant virtual objects in the overlay. The system will optimize data being sent to the computing device (e.g., mobile device) such that rendering is in real-time or near real-time. The system will use system resources either via an on-ground, aerial, or cloud-based system to render complex data sets and compute all 3D calculations. Augmented objects may include one or more types of animal data (e.g., including simulated data), or one or more derivatives from animal data, that provide information related to the one or more subjects. The augmented reality system may also include a terminal for a user to place a bet, evaluate or calculate a probability, view a prediction or possibility, and/or mitigate or prevent a risk. In a refinement, augmented reality system may also provide a recommendation and/or an action to be taken. The terminal and/or user's ability to place a bet, evaluate or calculate a probability, view a prediction or possibility, mitigate a risk, and/or take an action may be controlled via a variety of mechanisms including but not limited to audio control (e.g., voice control), a physical cue (e.g., head movement, eye movement, or hand gesture), a neural cue, a control found within the AR hardware, or with a localized device (e.g., mobile phone).

For wagering systems, the speculation system provides a number of novel opportunities with respect to offering dynamic wagering, which may be supported, at least in part, by one or more statistical models and/or artificial intelligence techniques. One or more new prop bets may be created dynamically based on user interaction with animal data. For example, and in the context of sports, if one or more users frequent animal data (e.g., view the animal data on an application) that features one or more similar characteristics (e.g., using heart rate as an example of the animal data users frequent, heart rate for Player A, or heart rate for all guards for Team B), the system may dynamically create a prop bet and target those one or more specific users (e.g., the bet could be: is Player A's max heart rate going to be above 180 bpm in the game; is the Average Heart Rate for all guards on Team B going to be less than 150 bpm for the last 5 minutes of the 4th quarter). Bets and products related to wagering and probability assessment (including risk mitigation, prevention products, recommendation products, etc.) can be created utilizing any type of animal data and/or its one or more derivatives. The system may also calculate odds, assign odds, modify odds, enhance odds, or utilize odds, either created internally or by one or more third parties, to accompany these bets. Personalized bets may also be created dynamically based on the one or more user interactions with animal data. For example, if a specific user frequents animal data associated with one or more parameters or characteristics (e.g., User A frequents historical heart rate data for set #2 across all tennis matches), a prop bet may be created by the system or a third party system interacting with that specific user that utilizes at least a portion of that animal data as part of the bet (e.g., a bet is created that enables a user to bet on whether Player A's Heart Rate will be above 180 bpm in Set #2 in Match #1, or whether Player B's Heart Rate will be above 186 bpm in Set #2 in Match #7). The system may also calculate odds, assign odds, modify odds, enhance odds, or utilize odds, either created internally or by one or more third parties, to accompany these one or more bets.

Dynamic pricing may also be introduced based on user interaction with animal data. For example, if there are one or more users that frequent animal data that share at least one characteristic (e.g., using heart rate as an example of the animal data users frequent, heart rate for Player A), the system may dynamically offer better pricing models (for products) and/or products for that specific one or more users around supplementary animal data related to the one or more characteristics the user is interested in. For example, instead of paying $10 for access to Player A's winning percentage when his heart rate is above 180 beats per minute, the system may adjust and charge $5.

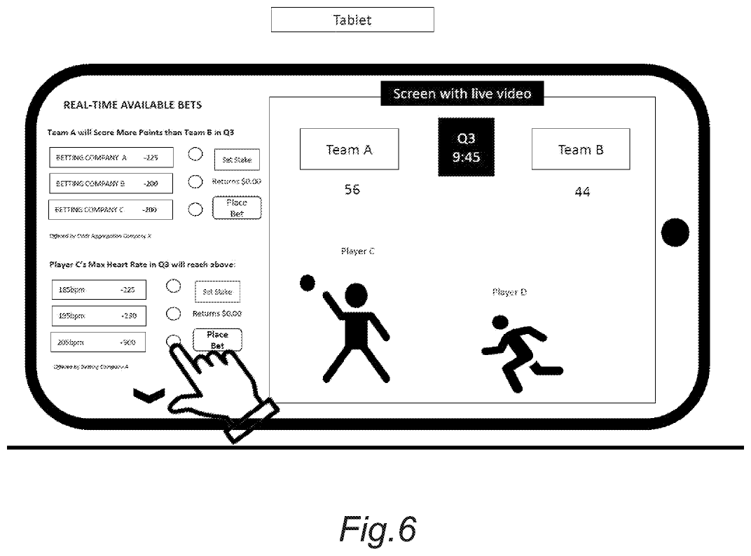

“Pop Up” wagering, which includes web advertising wagering or wagering within fan engagement applications (e.g., web application, mobile application for a smart device, virtual reality systems, augmented reality systems), may also be provided. FIG. 6 provides an example of a pop-up that may be displayed, or a wagering system that may be embedded, when a user is streaming a sporting event and the application is soliciting a user to place one or more bets or acquire one or more products. In a refinement, the pop up may be displayed as an overlay function on top of any given media (e.g., as a transparent display on top of any given media). Wagering within an advertisement, like an IFrame for example, may feature (1) at least one odds provided either directly or indirectly based on the animal data, (2) at least one bet type or other product either directly or indirectly based on the animal data, (3) at least one opportunity to place a bet, (4) at least one opportunity to acquire at least a portion of the animal data and/or its one or more derivatives, (5) at least one probability or prediction related to an outcome, (6) at least one recommendation to take an action, (7) at least one risk mitigation or prevention strategy, and/or (8) at least one promotion related to usage of the animal data. For example, a user may see an advertisement within a web page for life insurance. By asking an individual to upload, or provide access to, at least a portion of their animal data, the insurance company may create one or more predictive indicators that are utilized to evaluate an insurance premium for any given life insurance policy for any given subject (or group of subjects) in order to provide an adjusted or adjustable real-time or near real-time quote for insurance to the individual based at least in part on their animal data. In another embodiment represented in FIG. 5G, one or more bets or engagements may be featured on a web page or application for any specific subject, group of subjects, and the like (e.g., in the context of sports, a specific athlete, group, team, league, federation, or organization). When clicking a link (e.g., news article link) to view more content related to, for example, the subject or group of subjects (e.g., specific person, team, league), the system may offer one or more bets or products related to that subject (e.g., a specific player) or group of subjects within the website (e.g., via an IFrame). In a refinement, the act of scrolling the control mouse over the one or more subjects' names (e.g., player name, team name) in the article or content displayed via the web page or application, or touching a particular area of the content (e.g., the player name, team name) with a finger or other gesture on the screen, may trigger the system to offer one or more bets or products related to the area scrolled over or touched (e.g., if the control mouse is hovered over Player A's name, a bet for Player A appears). In a further refinement, using an audio-controlled (e.g., voice activated) or eye movement-controlled or sensing device may also trigger a bet or product to be presented. In another embodiment, “pop up” bets or products may occur within a digital destination (e.g., web page) targeting the specific content a user is reading about. For example, if a user is on a web page and reading about a specific team, one or more bets related to that team may pop up, and an ability to place one or more bets may be available within the digital destination or the user may be linked to another digital destination (e.g., another web page) to place a bet. These examples are not limited to sports and could be applicable to a variety of industries that may utilize a predictive indicator, computed asset, at least a portion of the animal data, and/or its one or more derivatives including healthcare, insurance, wellness, fitness, transportation, and the like. In another refinement, a virtual assistant may inform a user (e.g., text, audio, email) of one or more bets, bet types, products, recommendations, predictions, and/or types of data (e.g., predictive indicator) that may be of interest or available to a user based on one or more parameters a user sets (e.g., a user only wants to know about bets or products available for Player D on Team A related to his “energy level” in the 4^{th} quarter of home games) or content a user is interested in, which may be determined by the system or inputted by the user. In another refinement, a bet may appear within a virtual reality or augmented reality system alongside or integrated with other content. In yet another refinement, the animal data and/or the associated wager or product may be time-sensitive, with the system rejecting an ability to place a wager (or acquire a product) or placing an expiry time on a wager or product based upon one or more time conditions (e.g., the web page has been idle for more than 30 seconds). In these scenarios, animal data may render (e.g., via the display) either in a continuous, intermittent, or static manner. In the case of continuous or intermittent rendering, the content may be updated in real-time or near real-time. Upon clicking through to the third-party site, the publisher (e.g., the site or platform which featured the advertisement) and the data provider, which may consist of one or more parties including the sensor company, platform company, analytics company, individual from whom the data is derived, or owner of the data, may participate in a revenue share for the revenue generated from the click-through and/or user interaction with the data (e.g., a bet if the digital destination is programmed to enable a bet within the advertisement). If the user interacts with the advertisement to the point of purchase, the data provider may participate in further revenue share opportunities with the third-party site (e.g., mobile app).

As earlier referenced, prop bets are utilized to stimulate both betting volume and revenues. Examples include 10-minute markets used by a betting company: “3 or more corners in the next 10 minutes” or “Team A to score in the next 10 minutes.” These prop bets create new betting products/opportunities that provide a more diversified offering and differentiate bookmakers to ensure customer retention and stickiness. Animal data can be used as the driver for new and innovative bet types that will allow bookmakers to create prop bets with animal data that bettors have not yet seen. A few examples based on heart rate (which represent only a sample of potential bet types that the system may provide utilizing any type of animal data and/or its one or more derivatives) could include:
Player A's Average Heart Rate Beats Per Minute for Match w
Player A's Max Heart Rate Beats Per Minute for Match w
Player A's Average Heart Rate Beats Per Minute (BPM) for Game z of Match w will be higher than Player A's historical Average Heart Rate for Game z of Match w
Player A's Max Heart Rate (Max BPM) for Game z (e.g., game 4 of 2^{nd} set) of Match w will be higher than Player A's historical Max Heart Rate for all Game z's ever played (e.g., every game four for every second set played)
Player A's Average Heart Rate (BPM) for Match x will be higher than Player A's historical Average Heart Rate for all previous matches
Player A's Max Heart Rate (Max BPM) for Match x will be higher than Player A's historical Max Heart Rate for all previous matches
Player A's Max Heart Rate (Max BPM) will be higher than Player B's in the first n points of Set x
Player A's Average “Efficiency” for Game x (e.g., computation which may be established by examining how close Player A's heart rate is at any given time compared to his/her max heart rate. One way to calculate this computation may be Average Heart Rate divided by established Heart Rate Max) will be higher than Player B's in Game x
Player A's Max “Efficiency” for Set y (e.g., computation which may be established by Max heart rate divided by established Heart Rate Max) will be higher than Player B's in Set y
Player A's Average “Efficiency” in Match t (e.g., computation which may be established by Average Heart Rate divided by established Heart Rate Max) will be higher than Player B's in Match t
Player A's Max “Efficiency” for Match t (e.g., computation which may be established by Max heart rate divided by established Heart Rate Max) will be higher than Player B's in Match t
Player A will have a faster Recovery Rate (e.g., a computation which may be established by comparing player Efficiencies) than Player B in between Games x and y
Player A's resting Heart Rate (e.g., a computation which may be established by capturing resting BPM) will not be lower than a predetermined BPM in between Games x and y

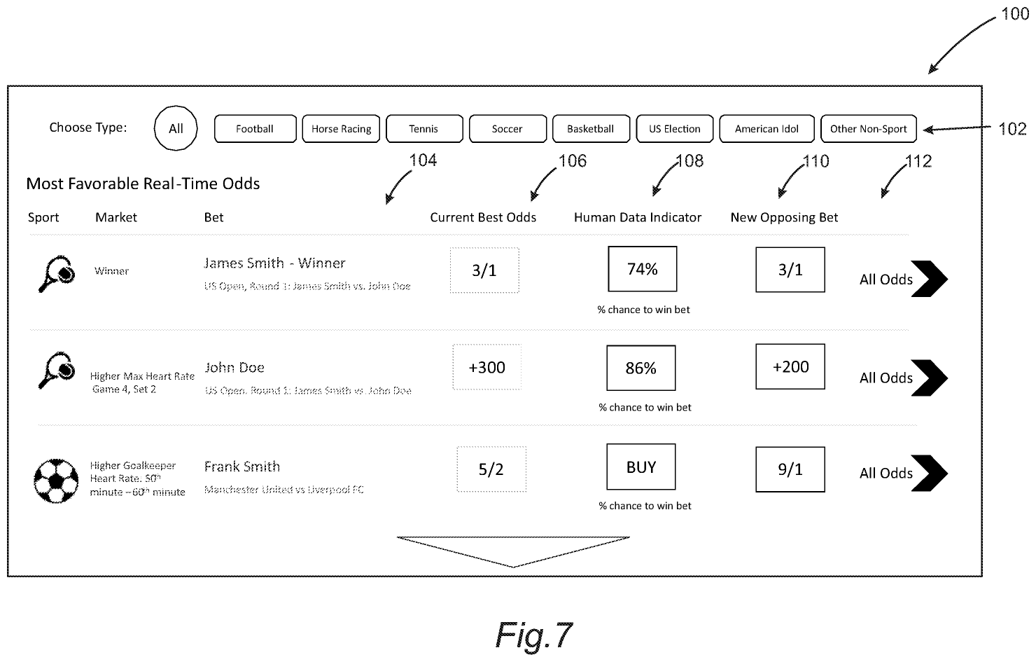

FIGS. 7 to 10 illustrate the functionality of the speculation system of FIG. 1 that can be deployed in a web page or in a window for a dedicated program or other application (e.g., smart device app). The term “window” will be used to refer to a web page and/or window for a program or computing device (e.g., smart device, smart phone, tablet, virtual reality headset, augmented reality headset, etc.) application. FIG. 7 is an example of a homepage of a wagering application. The wagering application can be owned/operated by the entity operating the speculation system (“speculation operator”) or by a third party that receives the Output Information from the speculation system. Homepage window 100 includes event selection section 102 that allows a user to choose an event (e.g., sport) on which to place a wager. Event selection section 104 lists a subset of the one or more wagers that can be placed (in this example, featuring the “Most Favorable Real-Time Odds”). Note that section 104 represents only a sample of potential wagers that the system may provide. Current best odds section 106 lists the best original odds offered by any third-party wagering system accepting wagers (“Wagering Entity”) for a particular event outcome (e.g., including prop bets). Odds section 106 may be updated (e.g., refreshed) as new odds information enters the system. In a refinement, the Wagering Entity is the operator of the speculation system. In another refinement, the Wagering Entity is part of the speculation system. In this FIG. 7 example, the most favorable odds offered on third-party sites (current best odds 106) for “James Smith to Win” are 3/1, so this particular bet—determined by the system to be favorable odds with a high indicator of success—is highlighted as a bet with the “Most Favorable Real-Time Odds”. The “Human Data Indicator” section 108, which is not limited to humans and can be derived from any animal, provides the predictive indicator derived from animal data (e.g., from one or more individuals or one or more groups of individuals such as a team) as set forth above. The “Human Data Indicator” section 108 is meant to demonstrate one way to convey a predictive insight that can provide a bettor with information that may give the bettor a greater understanding of the probability of an outcome occurring, the likelihood of a better winning any given bet, and/or provide confidence to a bettor to place one or more bets. In the example depicted in FIG. 7, the “Human Data Indicator” box 108 represents the percentage that a likely outcome will happen (e.g., 74% likely to win the bet based on current animal data, which may include both real-time and non-real-time data). This can be derived from any type of animal data and/or its one or more derivatives (e.g., heart rate, biological fluid data, location-based data like how far a player moves and where they move for each point, outcome data (e.g., whether they won the point or lost the point), historical data, and the like). Advantageously, the Human Data Indicator 108 can be updated in real-time or near real-time as new data is collected and analyzed. In FIG. 7, the predictive indicator is the % chance to win a bet, with a higher number indicating the likelihood of a more favorable bet outcome in section 104 to occur.

Still referring to FIG. 7, given that the speculation system can be programmed to generate one or more outcome probabilities or predictions, the speculation system can be operable to derive real-time or near real-time odds utilizing at least a portion of the Output Information for any given outcome. The “New Opposing Bet” section 110 provides the revised one or more odds that the speculation system is willing to offer to a bettor based on the system's use of the Output Information. In one example, “New Opposing Bet” refers to the odds being offered on the bet/market not being favored by the predictive indicator (e.g., Human Data Indicator) to win the bet. Given a potential position that any data may not be able to predict an outcome with 100% certainty, bettors may choose to ignore the data (e.g., predictive indicator) and place a bet with odds created or adjusted by the system (i.e., New Opposing Bet in this example), which may factor in at least a portion of the Output Information into the creation or adjustment of the odds. Oftentimes, these odds will be more favorable to a bettor who wishes to bet against the predictive indicator. The “New Opposing Bet” is meant to demonstrate one way to offer such new odds. Odds 110 may be offered by the entity operating the speculation system, by one or more third parties that receive the Output Information or odds information from the speculation system, or by one or more third parties that receive the Output Information or odds information from another third party. All Odds section 112 is a control element that displays All Odds web page or other information related to the animal data.

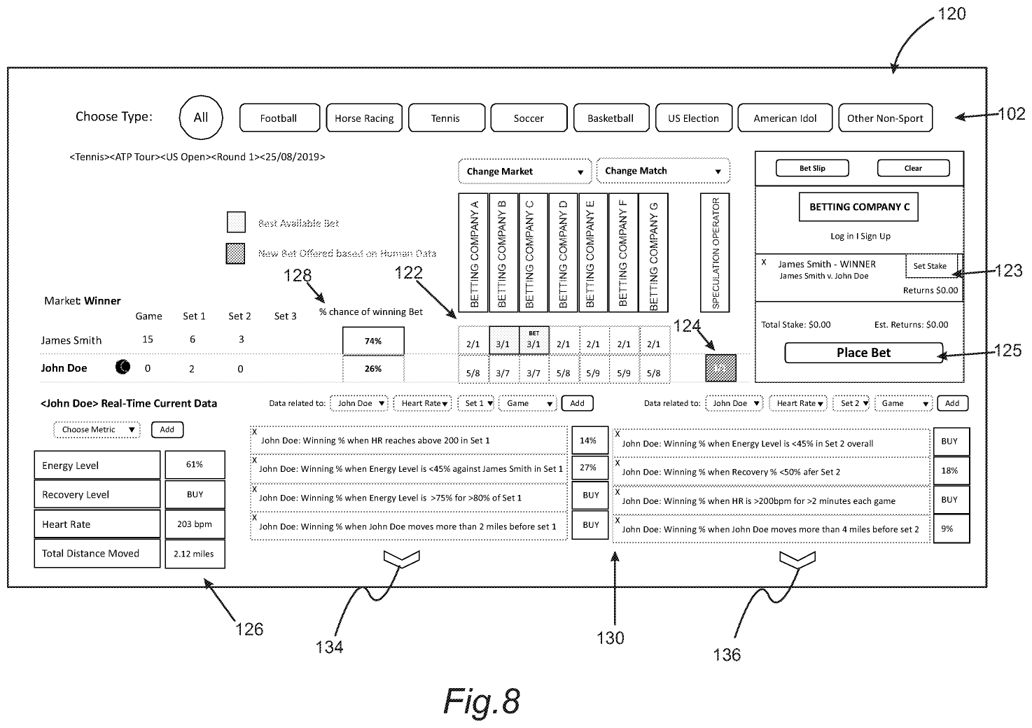

If the speculation operator also operates a wagering subsystem (either separately or as part of the speculation system), it may create or adjust its own odds, take one or more bets, and/or facilitate one or more transactions. However, an alternative scenario is illustrated in FIG. 8. As a system that collects and stores animal data and other information, the speculation operator could provide one or more users (e.g., wagerer) with third-party wagering odds information (e.g., third-party wagering odds information) for any selected event alongside animal data derived from the one or more individuals or groups of individuals (or other animal(s)) featured in the selected event. Advantageously, a user can access information related to the one or more animals or groups of animals (e.g., individual athletes, one or more teams of athletes) featured in the selected event prior to placing a bet, which can provide the wagerer with various types of animal data information as well as one or more probabilities, predictions, and/or possibilities related to the selected event. Characteristically, the data may be provided in real-time or near real-time. In betting window 120 of FIG. 8, the speculation operator aggregates odds information from third-party bookmakers and provides the user with the odds from relevant bookmakers as depicted in section 122. Odds can be displayed in multiple ways, provided for any given bet, and adjusted in real-time or near-real time. Section 122 enables the user to select one or more bets that may feature real-time odds adjustments. Advantageously, upon selection of a bet in section 122, a user can set their stake (e.g., how much the user wants to bet) via element 123 while selecting control element 125 to place the bet, which may be with a third-party wagering entity. Characteristically, the speculation system is operable to provide Output Information to the one or more users. Bets may be placed by the one or more users at any point in time while the bet is being offered. The type of data the system can provide may include, for example, animal data or a derivative, a recommendation of what bet to place, the likelihood of an outcome occurring, or a prediction related to any given outcome, which may include both animal and non-animal data. In some cases, the speculation operator may provide its own odds 124 upon which the speculation operator may accept one or more bets. Odds 124, which are the New Opposing Bet odds 110 from FIG. 7, will take into account at least a portion of the Output Information to understand the likelihood of any given outcome occurring. In most cases, the outcome-based data will be generated by running one or more simulations of the event. Based on the speculation operator's confidence in any given outcome, the speculation operator will generate odds 124 which will typically reflect a more favorable position for bettors that want to bet on the opposing view of what the speculation operator believes the one or more outcomes will be. In a refinement, odds 124 will be updated in real-time or near real-time as new Output Information or other data is collected and/or as one or more new simulations are run to create the new odds (which may or may not include updated Output Information but may include other information such as traditional stats, e.g., points won/lost, and other non-animal data). Typically, these odds will be favorable compared to odds offered by one or more other bookmakers. In another refinement, the speculation operator is an aggregator of belting odds so that a bettor can sec all the various odds on a single page. Once the speculation operator has collected the odds information, it can supplement the odds information via the animal data information to enable a bettor to see the most advantageous bet opportunity based on the data information the system provides. Advantageously, providing bettors with valuable information 126 like animal data (including computed assets and predictive indicators) at the right time can increase the frequency of bets placed and provide confidence to any bettor. This data may be provided to a bettor in a number of ways including on an ad-hoc basis (e.g., the bettor has the ability to acquire or purchase data by data type), by betting volume (e.g., the more bets or more money spent placing wagers, the more animal data information the bettor has access to), or on a subscription basis (e.g., the bettor may pay a fee per bet, per month, etc. for access to the animal data and/or its one or more derivatives, which can be inclusive of the computed assets and predictive indicators). Animal data may also enable bettors to form a stronger opinion about probable outcomes and encourage one or more impulsive bets by eliminating indecisiveness, giving the bettor the confidence they need to place another bet. Some examples are listed in section 130, which displays additional animal data-derived insights that provide a bettor with further information related to any given subject and/or any given bet. In a refinement, probability-based data may be provided to the speculation system by a third party (e.g., probability assessment system). Animal data could also provide bookmakers with the confidence to adjust odds dynamically, including in real-time or near real-time, which may increase the volume of bets placed.

The animal data, and in particular the human data, may be sold in a number of ways. The user can choose what animal data they want to consume (e.g., view) in real-time (or near real-time) and what non-real time data they want to consume based on the specific information they are targeting. The system may be operable to enable users to acquire (e.g., purchase) the data on a subscription basis, on an ad-hoc basis, on a per-sport basis, on a per transaction basis (e.g., per API call), on an animal data-type basis, or other basis. Given that users may have the ability to set one or more stakes and place one or more bets on a third-party site through the speculation operator's platform (thereby generating more revenue for the third party), the entity operating the speculation system may retain at least a portion of each bet that is placed on the third-party site.

Still referring to FIG. 8, the predictive indicator 128 (e.g., “% chance of winning Bet” percentage) may change dynamically based on one or more different factors. In one example, the predictive indicator 128 can change based on the type of animal data a user purchases from the system, or the type of subscription a user has which enables the user to access animal data, or more simply the type of animal data ingested and utilized by the speculation system. The frequency upon which the predictive indicator changes may also vary based upon a number of factors including the type of subscription or data package a bettor has purchased (e.g., displayed and updated every 5 seconds based on a new simulation being run every 5 seconds, or displayed and updated every 5 minutes based on a new simulation being run every 5 minutes), the number of simulations, the frequency upon which the speculation system takes one or more actions upon the Output Information, and the like. Depending on what is purchased, the Output Information may not be exhibited as a number (e.g., percentage). It may be shown in a number of ways including as a graph, a color (i.e., green might mean full power, red might mean very fatigued and out of energy), or other indices. It may also be communicated to a user in a number of ways including visually (as described above, which also may be integrated into a virtual reality or augmented reality offering and overlaid on top of an athlete or team), verbally (e.g., a virtual assistant providing audio related to the information and whether or not to place a bet), or physically (e.g., a user may have a smart watch that provides a notification and vibrates when the user receives the notification related to the data).

In a variation, the computing subsystem or the wagering system or the probability assessment system provides one or more users of data (e.g., bettor, bookmaker, insurance provider) with at least a portion of the predictive indicator, computed asset, animal data and/or its one or more derivatives in exchange for one or more users providing the computing subsystem or the wagering system or the probability assessment system with at least a portion of the consideration that is derived from: (1) the placing of a bet, the mitigation of a risk, the evaluation of a probability, the use of a prediction, a product being acquired or consumed, or providing a recommendation; (2) one or more users (e.g., bettors) as a result of one or more bets being won, one or more products being acquired or consumed, one or more risks being mitigated, or one or more probabilities being evaluated, one or more predictions being used, one or more recommendations being provided, or one or more actions being taken; or (3) one or more users (e.g., bookmakers, insurers of bets, insurers) who are offering the one or more bets or products upon which one or more bets are placed, one or more risks being mitigated, one or more probabilities are evaluated, one or more products are acquired or consumed, one or more predictions or recommendations offered, or one or more actions are taken. For example, the computing subsystem may offer a bettor the ability to obtain the predictive indicator, computed asset, animal data and/or its one or more derivatives related to any given bet being placed in exchange for the computing subsystem receiving a portion of the winnings should a bettor win the bet (e.g., the computing subsystem receives 1% of a winning bet placed in which a bettor obtained the predictive indicator). In another example, the computing subsystem may offer a bookmaker the ability to view the predictive indicator (e.g., human data indicator) or other animal data, or its one or more derivatives, related to a bet being offered in exchange for the computing subsystem receiving a percentage of the winnings (e.g., the computing subsystem receives n % of a bet in which the bookmaker wins based upon the bookmaker viewing the predictive indicator or other odds created based upon at least a portion of the animal data). In another example, an insurance company (e.g., automotive, health, life) may obtain the predictive indicator (e.g., human data indicator) or other animal data to determine the likelihood of an occurrence happening to one or more individuals (e.g., getting into a car accident, having a heart attack or stroke, experiencing a medical condition or event based on one or more animal data characteristics of the one or more individuals (e.g., physiological characteristics, age, medical conditions) in order to adjust the insurance premium paid by the individual, with the computing subsystem or the wagering system or the probability assessment system receiving consideration (e.g., via a portion of the monthly or yearly insurance fee paid by the individual). A life insurance company, for example, may want to obtain the predictive indicator to predict the life expectancy of a person with specific characteristics (e.g., age, weight, medical conditions) that exhibit specific biological characteristics. The computing subsystem may obtain a portion of the consideration paid by the one or more individuals to the insurance company in exchange for providing the predictive indicator, which may be accessible to the insurance company at any given time and updated in real-time or near real-time. One or more simulations may be run in order to generate the predictive indicator or other animal data. In a refinement, the user (e.g., the subject) may choose to provide at least a portion of the animal data or its one or more derivatives to the insurance company to have a premium adjusted, with the computing subsystem receiving consideration from the user (e.g., a portion of the projected savings).

In a refinement, the user of data (e.g., bettor, bookmaker, insurance provider) may have the ability to determine consideration (e.g., a monetary value or a percentage) the computing subsystem or the wagering system or the probability assessment system would receive in exchange for viewing the predictive indicator or other animal data or derivative thereof (e.g., odds of an event occurring), with the computing subsystem or the wagering system or the probability assessment system having the ability to accept, decline, or modify the consideration. For example, the bettor may want to obtain the predictive indicator, computed asset, or other animal data or its one or more derivatives for a consideration specified by the user (e.g., a user may want to give 1% of winnings or $20 on a $1000 bet placed). The computing subsystem or the wagering system or the probability assessment system may be programmed to accept, reject, or modify the offer being made by the bettor or bookmaker for access to the predictive indicator, computed asset, and/or other animal data or its derivative.

As previously described, for virtual bets like virtual horse racing or other gaming, speculation operators may use one or more statistical models or artificial intelligence techniques to create simulated animal data based at least in part on real animal data that users can engage with to understand the likely outcome of any given bet. The simulated animal data for virtual bets may also be used for the creation of new prop bets/markets for one or more virtual events.

Advantageously, the speculation system that utilizes animal data information can be configured in a number of different ways. Examples of such configurations include:

(1) a wagering subsystem where one or more users/bettors can define the bet and the wagering subsystem or one or more third parties can create one or more odds for it;

(2) a wagering subsystem where one or more users/bettors can define the bet and create one or more odds, and the wagering subsystem or one or more third parties can choose whether to accept the one or more odds and take the one or more bets;

(3) a wagering subsystem where one or more users/bettors can define the bet and create one or more odds, and the opportunity to acquire the rights to accept the one or more odds and the one or more bets are presented via an auction, a bidding opportunity, or a marketplace;

(4) a wagering subsystem where one or more users/bettors can define the bet, whereby the wagering subsystem or one or more third parties create the one or more odds and the one or more users/bettors can accept the one or more odds from the wagering subsystem or the one or more third parties; or

(5) a wagering subsystem where one or more users/bettors can define the bet and/or create one or more odds, and the wagering subsystem or one or more third parties can insure the bet and/or accept the risk on the payout to the bettor based on odds created by the wagering subsystem, the one or more third parties, or the user/bettor.

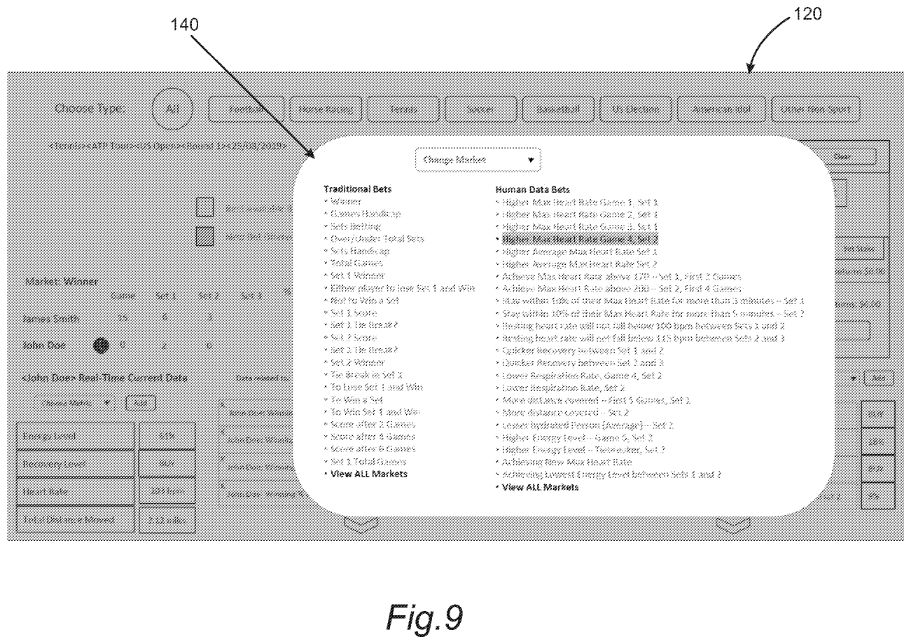

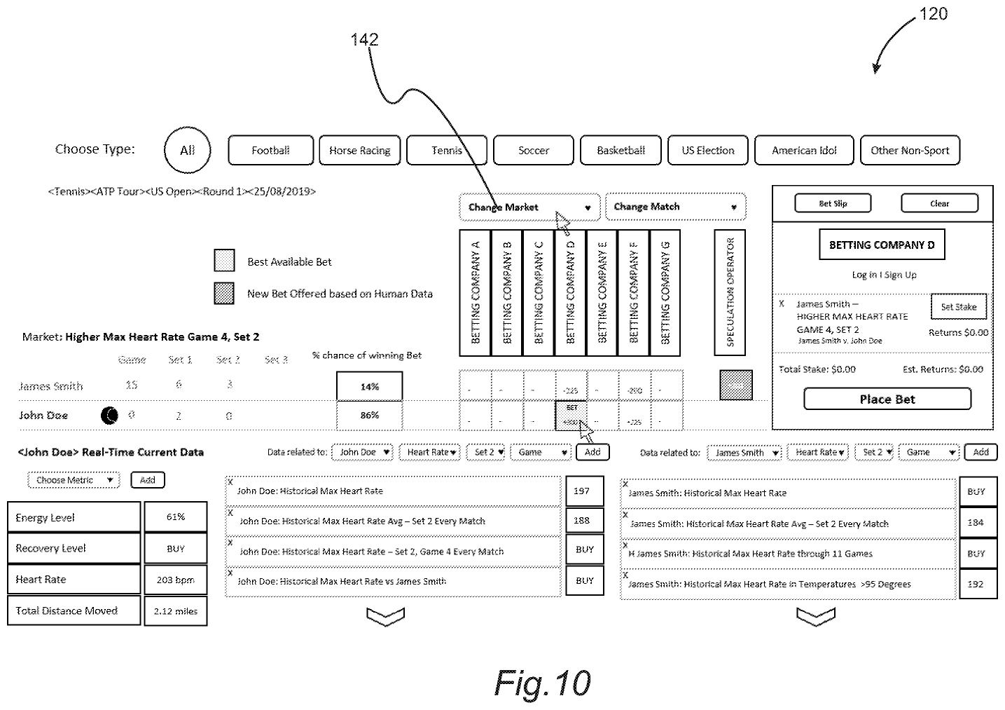

Advantageously, purchasing or acquiring additional data 130 in FIG. 8 may adjust the accuracy or precision of the Human Data Indicator, or the frequency upon which the Human Data Indicator is updated or rendered. Control elements 134 and 136 in FIG. 8 enable a user to scroll down to view more information in the event more data is purchased. FIG. 9 provides examples of the types of bets that could be offered. Betting section 140 provides specific examples of bets, and in particular proposition bets that can be provided to one or more users, which include bets based on the Output Information. Actuation of control element 142 in FIG. 10 causes window 140 in FIG. 9 to be displayed. With any singular type of animal data, the bets can become detailed and granular so that that a wagering subsystem can create a vast number of bets from the data. For example, and using heart rate as an example, a window 140 may display the following bet types:

Higher Max Heart Rate Game 1, Set 1

Higher Max Heart Rate Game 2, Set 1

Higher Max Heart Rate Game 3, Set 1

Higher Max Heart Rate Game 4, Set 2

Higher Average Max Heart Rate Set 1

Higher Average Max Heart Rate Set 2

Achieve Max Heart Rate above 170—Set 1, First 2 Games

Achieve Max Heart Rate above 200—Set 2, First 4 Games

Stay within 10% of their Max Heart Rate for more than 3 minutes—Set 1

Stay within 10% of their Max Heart Rate for more than 5 minutes—Set 2

Resting heart rate will not fall below 100 bpm between Sets 1 and 2

Resting heart rate will not fall below 115 bpm between Sets 2 and 3

FIG. 10 provides an example when a user chooses a new market (e.g., prop bet or other bet) via control element 142 in window 120. In the example of FIG. 10, a user has selected to place a bet on what player will have a “Higher Max Heart Rate Game 4, Set 2.” In this scenario, once the user chooses the market, the speculation system displays the odds provided by one or more wagering entities, as well as the odds provided by the speculation operator which is derived utilizing at least a portion of the Output Information. The speculation system then provides at least a portion of the animal data as part of one or more data points (e.g., predictive indicator) that enables a user to see one or more forecasts, predictions, or recommendations (e.g., what the chances of winning the bet am at any given time based on the animal data collected). For example, the speculation system 120 provides a predictive indicator in FIG. 10 that John Doe has an 86% chance of having a higher heart rate this game. This may be based on one or more data points that a user searches for and acquires (e.g., purchases) 126. Using this example in the illustration, “Historical Max Heart Rate,” “Historical Max Heart Rate Avg—Set 2 Every Match,” “Historical Max Heart Rate—Set 2, Game 4 Every Match,” and/or “Historical Max Heart Rate vs.” the particular opponent. In a refinement, multiple predictive indicators may be generated and displayed simultaneously. A bettor can also search for and acquire additional information on each player that will give the bettor the confidence to place a bet. Given speculation entity's predictive indicator labeled “Human Data indicator,” it may be willing to provide bettors with more favorable odds if a bettor desires to wager on the system's projected “loser.”

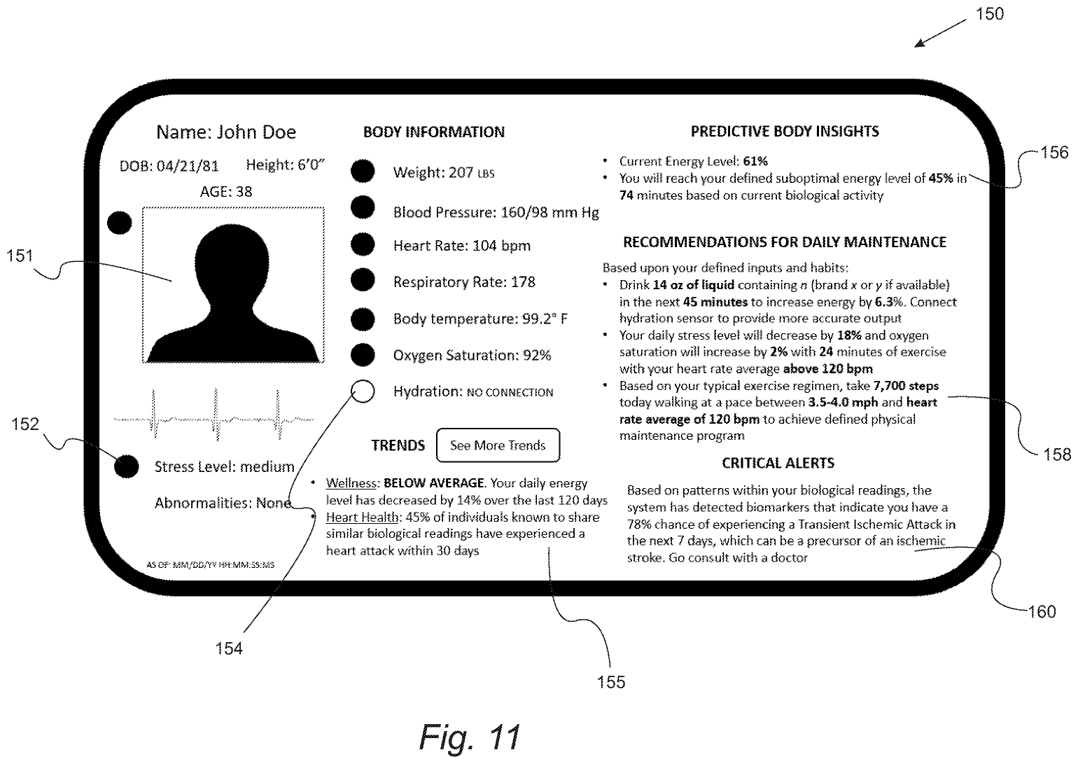

FIG. 11 illustrates the functionality of the speculation system of FIGS. 1, 2, and 3 that can be deployed in a web page or in a window for a dedicated health program or other health application for a targeted subject, a plurality of targeted subjects, or one or more groups of targeted subjects. Health system 150 includes one or more outputs along with other animal data related to the one or more targeted subjects. Health system 150 can include window 151, which may be a static picture of the targeted subject or a video (e.g., live or delayed depending on the use case) of the targeted subject. In a variation, the one or more targeted subjects may be able to see another one or more animals (e.g., humans) within the same window (e.g., a patient seeing a doctor or medical professional within a telehealth platform, or a client seeing their rehabilitation specialist in the same window within their rehabilitation program). Additional functionality (e.g., whether or not a video camera is connected to the system, whether or not the camera is turned on or off) may also be displayed. The system may be operable to detect whether a sensor is connected to the system (which may be displayed as connection notification element 152) or disconnected to the system (which may be displayed as connection notification element 154). Animal information with real-time or near real-time outputs can be displayed along with information from which one or more predictions, probabilities, or possibilities can be calculated, computed, derived, extracted, extrapolated, modified, enhanced, estimated, evaluated, inferred, deduced, established, determined, observed, communicated, or actioned upon. Section 156 provides the one or more predictive indicators derived from the animal data, while section 158 provides one or more recommendations based on predictions and probabilities established by the system. Trends section 155 can provide real-time or pre-defined timed trends related to the live and historical biological signals and readings along with other animal data. Additional fields may be added based upon the animal data. Advantageously, the system may be programmed to identify one or more critical alerts 160 that requires attention from the one or more subjects and/or the one or more users of the system (e.g., a medical professional utilizing the system and monitoring a targeted subject) based on the one or more outputs. The one or more critical alerts may be set with a predefined threshold by the individual or administrator (e.g., if the likelihood of something occurring is greater than n %, it is communicated as a critical alert) to alert one or more users of a potential issue related to one or more signals or readings. Characteristically, the system may be set up to utilize one or more artificial intelligence techniques to correlate data sets to identify known biological-related issues from one or more targeted individuals or groups of targeted individuals, as well as identify hidden patterns within the one or more data sets to identify biological-related issues based upon the collected data. This may include finding entirely new patterns within data that has never previously been correlated with known issues, or finding new patterns amongst one or more data sets that may identify new issues.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A speculation system comprising:
one or more source sensors that collect animal data from one or more targeted individuals wherein the animal data is transmitted electronically;
a computing subsystem that receives the animal data, at least a portion of the animal data being transformed by the computing subsystem or the one or more source sensors into at least one computed asset assigned to a selected targeted individual or group of targeted individuals, the one or more source sensors or the computing subsystem being operable to transform the at least one computed asset into a predictive indicator, the computing subsystem being further operable to provide the predictive indicator, the at least one computed asset, and/or at least a portion of the animal data to one or more users; and
a transmission subsystem providing transmission of the animal data to the computing subsystem.

2. The speculation system of claim 1 wherein the one or more source sensors consist of at least one biosensor.

3. The speculation system of claim 1 wherein the at least one computed asset includes one or more numbers, a plurality of numbers, metrics, insights, graphs, charts, or plots that are derived from at least a portion of the animal data.

4. The speculation system of claim 3 wherein the at least one computed asset includes one or more signals or readings from non-animal data.

5. The speculation system of claim 1 wherein the predictive indicator is a calculated computed asset from at least a portion of the animal data.

6. The speculation system of claim 5 wherein the predictive indicator includes one or more signals or readings from non-animal data.

7. The speculation system of claim 5 wherein the predictive indicator is comprised of a plurality of predictive indictors.

8. The speculation system of claim 5 wherein at least a portion of the predictive indicator is derived from or related to, at least in part, a group consisting of a targeted individual, multiple targeted individuals, a targeted group comprised of multiple targeted individuals, or multiple targeted groups comprised of multiple targeted individuals.

9. The speculation system of claim 5 wherein the predictive indicator is a composite calculated from two or more signals or readings from one or more source sensors.

10. The speculation system of claim 5 wherein the predictive indicator is calculated from the at least one computed asset that includes biological data selected from the group consisting of facial recognition data, eye tracking data, blood flow data, blood volume data, blood pressure data, biological fluid data, body composition data, biochemical composition data, biochemical structure data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, thoracic electrical bioimpedance data, or a combination thereof.

11. The speculation system of claim 5 wherein at least a portion of the predictive indicator is used either directly or indirectly: (1) as a market upon which one or more wagers are placed or accepted; (2) to create, modify, enhance, acquire, offer, or distribute one or more products; (3) to evaluate, calculate, derive, modify, enhance, or communicate one or more predictions, probabilities, or possibilities; (4) to formulate one or more strategies; (5) to take one or more actions; (6) to mitigate or prevent one or more risks; (7) as one or more readings utilized in one or more simulations, computations, or analyses; (8) as part of one or more simulations, an output of which directly or indirectly engages with one or more users; (9) to recommend one or more actions; (10) as one or more core components or supplements to one or more mediums of consumption; (11) in one or more promotions; or (12) a combination thereof.

12. The speculation system of claim 1 wherein the computing subsystem uses one or more outputs from the computing subsystem either directly or indirectly: (1) as a market upon which one or more wagers are placed or accepted; (2) to accept one or more wagers; (3) to create, enhance, modify, acquire, offer, or distribute one or more products; (4) to evaluate, calculate, derive, modify, enhance, or communicate one or more predictions, probabilities, or possibilities; (5) to formulate one or more strategies; (6) to take one or more actions; (7) to mitigate or prevent one or more risks; (8) as one or more signals or readings utilized in one or more simulations, computations, or analyses; (9) as part of one or more simulations, an output of which directly or indirectly engages with one or more users; (10) to recommend one or more actions; (11) as one or more core components or supplements to one or more mediums of consumption; (12) in one or more promotions; or (13) a combination thereof.

13. The speculation system of claim 12 wherein the one or more direct or indirect uses by the computing subsystem are dynamic, at least in part, and based upon one or more user interactions with the one or more outputs from the computing subsystem.

14. The speculation system of claim 12 wherein the market or wager includes at least one of, a proposition bet, spread bet, a line bet, a future bet, a parlay bet, a round-robin bet, a handicap bet, an over/under bet, a full cover bet, or a teaser bet.

15. The speculation system of claim 12 wherein the one or more outputs from the computing subsystem are dynamically created, modified, or enhanced by the computing subsystem.

16. The speculation system of claim 15 wherein creation, modification, or enhancement of the one or more outputs is based on or derived from, at least in part, one or more user interactions with the predictive indicator, the at least one computed asset, and/or the animal data.

17. The speculation system of claim 15 wherein at least a portion of dynamically created, modified, or enhanced one or more outputs are utilized either directly or indirectly: (1) as a market upon which one or more wagers are placed or accepted; (2) to create, modify, enhance, acquire, offer, or distribute one or more products; (3) to evaluate, calculate, derive, modify, enhance, or communicate one or more predictions, probabilities, or possibilities; (4) to formulate one or more strategies; (5) to take one or more actions; (6) to mitigate or prevent one or more risks; (7) as one or more signals or readings utilized in one or more simulations, computations, or analyses; (8) as part of one or more simulations, an output of which directly or indirectly engages with one or more users; (9) to recommend one or more actions: (10) as one or more core components or supplements to one or more mediums of consumption; (11) in one or more promotions; or (12) a combination thereof.

18. The speculation system of claim 1 wherein the computing subsystem provides one or more data outputs to one or more systems.

19. The speculation system of claim 18 wherein the predictive indicator is created, modified, enhanced by the one or more systems.

20. The speculation system of claim 18 wherein the one or more systems are operable to utilize at least a portion of the one or more data outputs either directly or indirectly: (1) as a market upon which one or more wagers are placed or accepted; (2) to accept one or more wagers; (3) to create, enhance, modify, acquire, offer, or distribute one or more products; (4) to evaluate, calculate, derive, modify, enhance, or communicate one or more predictions, probabilities, or possibilities; (5) to formulate one or more strategies; (6) to take one or more actions; (7) to mitigate or prevent one or more risks; (8) as one or more signals or readings utilized in one or more simulations, computations, or analyses; (9) as part of one or more simulations, an output of which directly or indirectly engages with one or more users; (10) to recommend one or more actions; (11) as one or more core components or supplements to one or more mediums of consumption; (12) in one or more promotions; or (13) a combination thereof.

21. The speculation system of claim 1 wherein the computing subsystem provides the same or substantially similar one or more outputs to a plurality of users.

22. The speculation system of claim 1 wherein the one or more outputs of the computing subsystem are synchronized with one or more non-animal data readings.

23. The speculation system of claim 1 wherein the one or more outputs of the computing subsystem are synchronized with one or more mediums of consumption.

24. The speculation system of claim 1 wherein the computing subsystem is operable to receive groups of animal data from a single targeted individual or multiple targeted individuals.

25. The speculation system of claim 1 wherein the computing subsystem is operable to gather information from one or more source sensors by communicating directly with the one or more source sensors, its associated cloud, or a native application associated with the one or more source sensors.

26. The speculation system of claim 25 wherein the computing subsystem is operable to manage the one or more source sensors, and one or more data streams from the one or more source sensors, by at least one characteristic from the group consisting of; organization, sensor type, sensor parameter, data type, data quality, time stamp, location, activity, the targeted individual, groupings of targeted individuals, and data reading.

27. The speculation system of claim 1 wherein the computing subsystem is operable to communicate with a plurality of source sensors on the targeted individual or one or more source sensors on multiple targeted individuals simultaneously.

28. The speculation system of claim 1 wherein the transmission subsystem enables the one or more source sensors to transmit data wirelessly for real-time or near real-time communication.

29. The speculation system of claim 1 wherein the transmission subsystem communicates with the one or more source sensors utilizing one or more transmission protocols.

30. The speculation system of claim 1 wherein the computing subsystem synchronizes communication with one or more data signals or readings from multiple sensors that are in communication with the computing subsystem.

31. The speculation system of claim 1 wherein the transmission subsystem includes a transmitter and a receiver, or a combination thereof.

32. The speculation system of claim 1 wherein the transmission subsystem includes an on-body or aerial transceiver that optionally acts as another sensor on or above the targeted individual, the on-body or aerial transceiver being operable to communicate with other one or more sensors on one or more targeted individuals.

33. The speculation system of claim 1 wherein the animal data is synchronized, time-stamped, and tagged with information related to the one or more targeted individuals from which the animal data is collected and the one or more source sensors, which includes at least one characteristic of the one or more source sensors.

34. The speculation system of claim 1 wherein the animal data includes metadata that identifies one or more characteristics of the animal data and the one or more source sensors.

35. The speculation system of claim 1 further comprising of a wagering system or probability assessment system, or a combination thereof.

36. The speculation system of claim 35 wherein the computing subsystem or the wagering system or the probability assessment system execute one or more actions on the animal data selected from the group consisting of normalizing, time stamping, aggregating, tagging, storing, manipulating, denoising, productizing, enhancing, organizing, visualizing, analyzing, summarizing, replicating, synthesizing, anonymizing, synchronizing, or distributing the animal data.

37. The speculation system of claim 35 wherein the computing subsystem or the wagering system or the probability assessment system: (1) communicates directly with one or more systems to monitor, receive, and record at least one request for the predictive indicator, the at least one computed asset, and/or the animal data; (2) provides the one or more users requesting access to the predictive indicator, the at least one computed asset, and/or the animal data with an ability to make one or more requests for data; and (3) is operable to send and/or receive data.

38. The speculation system of claim 35 wherein the computing subsystem or the wagering system or the probability assessment system associates at least one request for the predictive indicator, the at least one computed asset, and/or the animal data with at least one user, group of users, or class of users.

39. The speculation system of claim 35 wherein the computing subsystem or the wagering system or the probability assessment system is operable to allow one or more users to select at least one characteristic upon which animal data, the at least one computed asset, and/or the predictive indicator is provided.

40. The speculation system of claim 35 wherein the computing subsystem or the wagering system or the probability assessment system generates simulated data derived from at least a portion of the predictive indicator, the at least one computed asset, and/or the animal data of the one or more targeted individuals or groups of targeted individuals.

41. The speculation system of claim 40 wherein the simulated data is generated utilizing one or more signals or readings from non-animal data as one or more inputs.

42. The speculation system of claim 40 wherein simulated data is generated utilizing an artificial intelligence technique.

43. The speculation system of claim 42 wherein the artificial intelligence technique includes one or more trained neural networks.

44. The speculation system of claim 40 wherein the computing subsystem or the wagering system or the probability assessment system utilizes at least a portion of the simulated data either directly or indirectly: (1) as a market upon which one or more wagers are placed or accepted; (2) to accept one or more wagers; (3) to create, enhance, modify, acquire, offer, or distribute one or more products; (4) to evaluate, calculate, derive, modify, enhance, or communicate one or more predictions, probabilities, or possibilities; (5) to formulate one or more strategies; (6) to take one or more actions; (7) to mitigate or prevent one or more risks; (8) as one or more signals or readings utilized in one or more simulations, computations, or analyses; (9) as part of one or more simulations, an output of which directly or indirectly engages with one or more users; (10) to recommend one or more actions; (11) as one or more core components or supplements to one or more mediums of consumption; (12) in one or more promotions; or (13) a combination thereof.

45. The speculation system of claim 40 wherein the computing subsystem or the wagering system or the probability assessment system applies at least a portion of the simulated data, either directly or indirectly, to create, enhance, or modify the predictive indicator, at least one computed asset, and/or animal data.

46. The speculation system of claim 45 wherein at least a portion of created, enhanced, or modified predictive indicator, the at least one computed asset, and/or animal data is utilized either directly or indirectly: (1) as a market upon which one or more wagers are placed or accepted; (2) to create, modify, enhance, acquire, offer, or distribute one or more products; (3) to evaluate, calculate, derive, modify, enhance, or communicate one or more predictions, probabilities, or possibilities; (4) to formulate one or more strategies; (5) to take one or more actions; (6) to mitigate or prevent one or more risks; (7) as one or more signals or readings utilized in one or more simulations, computations, or analyses; (8) as part of one or more simulations, an output of which directly or indirectly engages with one or more users; (9) to recommend one or more actions; (10) as one or more core components or supplements to one or more mediums of consumption; (11) in one or more promotions; or (12) a combination thereof.

47. The speculation system of claim 1 wherein the animal data is grouped into one or more classifications with each classification having an associated computed asset or value.

48. The speculation system of claim 1 wherein upon sending the predictive indicator, the at least one computed asset, and/or animal data to another source, the computing subsystem records one or more characteristics of the predictive indicator, the at least one computed asset, and/or animal data provided as part of its one or more distributions.
